(19) United States
(12) Patent Application Publication
(10) Pub. No.: US 2017/0333941 A1
(43) Pub. Date: Nov. 23, 2017

(54) HIGH ENERGY EFFICIENCY PHASE CHANGE DEVICE USING CONVEX SURFACE FEATURES

(71) Applicant: President and Fellows of Harvard College, Cambridge, MA (US)

(72) Inventors: Kyoo-Chul PARK, Cambridge, MA (US); Philseok KIM, Waltham, MA (US); Joanna AIZENBERG, Boston, MA (US)

(21) Appl. No.: 15/522,680

(22) PCT Filed: Oct. 28, 2015
(86) PCT No.: PCT/US15/57879

§ 371 Date: Apr. 27, 2017

Related U.S. Application Data
(60) Provisional application No. 62/069,591, filed on Oct. 28, 2014.

(51) Int. Cl. B05D 5/08; F28B 1/00; C09K 5/04; F28F 13/18; F28F 13/04; C07C 19/00

(52) U.S. Cl. CPC B05D 5/08; F28F 13/187; F28F 13/04; F28B 1/00; C09K 5/044; C07C 19/00; B05D 2202/25

(57) ABSTRACT

Sub-micrometer to centimeter scale rough symmetric and asymmetric structures are incorporated onto objects (e.g. tubes and fms). Asymmetric and hierarchically structured slippery structures can be applied to a broad range of materials and shapes of surfaces for manufacturing heat exchangers, dew harvesting devices, desalination devices, de-humidifiers, distillation towers, evaporation coils, anti-cavitation coatings, etc.

### RELATED APPLICATIONS

This application claims the benefit of priority under 35 U.S.C. §119(e) to co-pending U.S. application Ser. No. 62/069591, filed on Oct. 28, 2014, the contents of which are incorporated by reference.

### STATEMENT REGARDING FEDERALLY SPONSORED RESEARCH OR DEVELOPMENT

The present invention was made with United States government support under Grant No. DE-AR0000326 awarded by the Department of Energy/ARPA-E. The United States government may have certain rights in this invention.

### INCORPORATION BY REFERENCE

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND

Phase change (e.g., condensation, vaporization, sublimation, frosting, melting, freezing) occurs on a surface if the surface is cooled or heated below or above the saturation temperature at a given pressure. For example, the condensing phase may grow on the surface as a liquid film and/or as droplets or islands of liquid. Heat transfer through phase change is an important process used in power plant condensers, water harvesters, desalination plants, distillation towers (e.g., hydrocarbons, polyolefins, hydrofluorocarbon, etc.), and thermal/humidity control systems in building. In many applications, it is useful to remove or collect the material after phase change for higher heat transfer efficiency as well as simply collecting the condensates for other use. However, the growth of droplets on condensers to reach the diameter of spontaneous removal is slow due to low rate of vapor diffusion and subsequent droplet coalescence, and the spontaneous shedding of strongly pinned condensates can only happen when the droplets grow to very large sizes. As a result, thick thermally insulating condensate films/droplets persist on low-temperature walls leading to tremendous energy inputs, greenhouse gas emission and excessive usage of coolant. For many industrial applications, it is therefore useful to inhibit or prevent the filmwise buildup of condensing liquid (due to its severe effect on heat transfer) by promoting and accelerating droplet shedding.

Superhydrophobic surfaces (SHS), which make use of an entrapped air layer to reduce the friction at the solid surface, have been believed to be the most promising technique for higher droplet growth and fast shedding of condensates. Despite more than a decade of intense research, these surfaces are, however, still plagued with inevitable problems: the air layer inhibits heat transfer due to its low thermal conductivity; fully wetted droplets trapped in the structured surface produce highly pinned condensates (or different phase materials, e.g., ice, after phase change); the surfaces get easily contaminated with both organic and inorganic particles; cannot self-heal or self-clean, and are expensive to produce.

Slippery Liquid-Infused Porous Surfaces or “SLIPS” consist of a film of lubricating liquid locked in place by a nano/microporous substrate. See, FIG. 17. By proper choice of a lubricant, surface functionalization and nano/microtextured surfaces, the lubricant can be immobilized within the space between and form a conformal overlayer of the nano/micro-asperities producing a slippery liquid interface. The liquid surface is intrinsically smooth and defect-free down to the molecular scale; provides immediate self-repair by wicking into damaged sites in the underlying substrate; and can be chosen to repel immiscible liquids of virtually any surface tension.

In contrast to superhydrophobic surfaces, SLIPS (or lubricated highly slippery surfaces) have been shown to exhibit negligible contact angle hysteresis when in contact with various condensates, excellent thermal contact due to the absence of the intermediate air layer, and in-plane smooth shedding, particularly over a broad range of temperature and relative humidity or saturation pressure conditions. Previous efforts (Scientific Reports 3, 1988 (2013), and ACS Nano 6, 10122 (2012)) that apply SLIPS to condensation are limited to simple attempts of using SLIPS in humid conditions on flat surfaces.

Improved or more efficient methods to prevent film buildup and promote droplet shedding are desired.

### SUMMARY

In one aspect, the invention includes introducing nanometer to centimeter scale symmetric and asymmetric, raised or recessed structures onto objects (e.g., metal tubes and fins, polymeric tubes, carbon, cement surfaces, etc.) and applying the SLIPS treatment based on uniform nano/microtextures (“nano/micro-SLIPS”) on such a topographically changed substrate. This creates hierarchically structured SLIPS surfaces.

In certain embodiments, the topographical features are convex surface structures, that is, the raised structures in which at least a portion exhibits a curved surface having a radius of curvature that is not infinite. In certain embodiments, a portion of the raised topographical features contains curved surfaces. These structures can induce accelerated localized nucleation and growth of condensate.

In certain embodiments, the topographical features are asymmetric, and the structural anisotropy facilitates fast coalescence and directional shedding of condensate. In one or more embodiments, the raise feature can include an inclined or sloped side that serves as a ‘ramp’ to direct the shedding of the condensate. When combined with the intrinsic ability of nearly friction-free SLIPS surfaces to induce high mobility of droplets, these two geometrical modifications (convexity and anisotropy of additional surface features) collectively enhance the condensation efficiency. Asymmetric and hierarchically structured slippery surfaces (“SLIPS-A”) used in this invention can be applied to a broad range of materials and shapes of surfaces for manufacturing phase change (e.g., condensation, vaporization, sublimation, deposition, melting, freezing)-based devices including heat exchangers, dew harvesting devices, desalination devices, distillation towers (e.g., hydrocarbons, polyolefins, hydrofluorocarbon, etc.), dehumidifiers, evaporation coils, anti-cavitation coatings, vapor deposition devices, etc.

In one aspect, a phase change-based device includes a thermally conductive substrate having a plurality of raised or recessed macro-features having a convex surface, wherein the macro-features are coated with a slippery coating comprised of a nano- to micro-scale roughened surface and a lubricating liquid stably immobilize in, on and over the roughened surface, wherein the macro-features and the slippery coating, in combination, promotes droplet, solid or bubble formation, growth, and removal of a phase of a phase-change material.

As used herein the ‘macro-feature’ has a dimension on a scale that is at least 1 order of magnitude, and in some cases 2 orders of magnitude, and in some cases 3 or greater orders of magnitude greater than the nano- to micro-scale roughened surface features. Thus, by way of example, a device according to one or more embodiments, having a raised surface features with dimensions (e.g., height, width and length) on the order of 500 μm-1 mm can have roughened surface with asperities or pores on the order of 200-500 nm (more than a 10³-fold difference in dimensions).

In one aspect, a phase change-based device includes a substrate comprising a plurality of macro-scale raised or recessed features having a convex surface, wherein the geometry of the feature promotes droplet, solid or bubble formation and accelerated growth on the apex of the raised feature, and removal of a phase of a phase-change material.

In one or more embodiments, the surface with a plurality of macro-scale features is coated with a slippery coating comprised of a lubricating liquid stably immobilize in, on and over the surface to promote accelerated removal of the phase change material.

In any of the preceding embodiments, the device further includes a slope that transitions from an apex of the raised feature (or nadir of the recessed feature) tangentially to the substrate.

In any of the preceding embodiments, the raised or recessed features in combination with the slope forms an asymmetric feature to provide directional removal.

In any of the preceding embodiments, the raised or recessed features in combination with the slope forms a ramp around at least a portion of the raised feature to provide droplet, solid or bubble removal in more than one direction.

In any of the preceding embodiments, the raised features include at least one rounded edge.

In any of the preceding embodiments, the raised features include a plateau integral with at least one rounded edge.

In any of the preceding embodiments, the plateau spans a pair of edges of the raised features.

In any of the preceding embodiments, the plateau is on the order of 100 nm to 10 cm.

In any of the preceding embodiments, the raised features include a cone, a hemisphere, a hemi-ellipse, a hemicylinder, pyramids, or bumps of irregular shape.

In any of the preceding embodiments, the raised features include a flat upper surface and are selected from the group consisting of one or more of cubes, rectangular prisms, cylindrical columns, truncated cones, truncated pyramids and or truncated bumps of irregular shapes.

In any of the preceding embodiments, the width of the slope increases from the point distal to the substrate to the substrate surface.

In any of the preceding embodiments, the recessed features is a groove.

In any of the preceding embodiments, the groove tapers from a first wide width to a second narrow width.

In any of the preceding embodiments, the groove has an inclined floor that slopes upward tangentially to the substrate surface.

In any of the preceding embodiments, the groove is in fluid contact with a reservoir holding lubricating liquid.

The In any of the preceding embodiments, the grooves are arranged to form a plurality of intersecting channels on the substrate.

In any of the preceding embodiments, the features have a width in the range of 100 nm to 10 cm, or the features are on the order of 0.1 mm to 1 cm.

In any of the preceding embodiments, the surfaces of the substrate and the macro-scale features comprise a nano-scale to micro-scale roughened surface.

In any of the preceding embodiments, the roughened surface comprises asperities, texture or porosity in the range of 5 nm to 100 μm, or the roughened surface comprises asperities, texture or porosity in the range of 10 nm to 5 μm.

In any of the preceding embodiments, the roughened surface is integral with the feature surface.

In any of the preceding embodiments, the roughened surface is coated over the feature surface.

In any of the preceding embodiments, the roughened surface is a porous metal oxide.

In any of the preceding embodiments, the macro-scale features have a roughness of R=0, that is, essentially flat.

In any of the preceding embodiments. The device of claim 26, wherein the surface of the device and the features are made of a polymer that can be swollen with the lubricating liquid.

In any of the preceding embodiments, the surface of the device is chemically functionalized to render surface compatible with the lubricating liquid.

In any of the preceding embodiments, the lubricating liquid is a hydrophobic or omniphobic liquid.

In any of the preceding embodiments, the lubricating liquid is selected from the group of hydrocarbon oils, partially or fully fluorinated oils, food-grade oils, mineral oils, silicone oils or ionic liquids.

In any of the preceding embodiments, the features are made of the same material as the substrate.

In any of the preceding embodiments, the features and the substrate are made of different materials.

In any of the preceding embodiments, the features are part of a film attached to the substrate.

In any of the preceding embodiments, the features are arranged in an array.

In any of the preceding embodiments, are arranged in an array of rows, and for example, rows of the array are staggered.

In any of the preceding embodiments, the features are arranged randomly on the surface.

In any of the preceding embodiments, the features are the same or different in shape, size, steepness of the slope, shape of the slope and direction of the slope.

In any of the preceding embodiments, the substrate is thermally conductive, and for example, the thermally conductive material includes a metal or the thermally conductive material comprises a metal mesh embedded in a polymer substrate.

In any of the preceding embodiments, the substrate with raised features is made of a deformable material.

In any of the preceding embodiments, the device can be reversibly deformed to switch, induce and additionally guide the droplet growth and removal.

In any of the preceding embodiments, the device is in the shape of a pipe or coil.

In any of the preceding embodiments, the device further includes a reservoir for supplying lubricating liquid to the device.

The device further includes microchannels that are aligned and perpendicular to the axis of the pipe to facilitate transport of lubricating liquid from a reservoir through the microchannels.

In any of the preceding embodiments, the features possess a radius of curvature R_{bump} of hemispherical features (or the width of asymmetric features W) and a feature to feature spacing Pₚₐₜₜₑᵣₙ and the features are positioned to provide a Pₚₐₜₜₑᵣₙ/R_{bump} (or Pₚₐₜₜₑᵣₙ/W) in the range of 1.1 to 100, or P/R is in the range of 2.5-100.

In any of the preceding embodiments, the device further includes a heat sink or coolant for removing adsorbed heat from the device.

In any of the preceding embodiments, the device forms at least a portion of a phase change-based device in thermal power plant condensers, water harvesters, desalination plants, distillation towers (e.g., water, hydrocarbons, polyolefins, hydrofluorocarbons), building thermal/humidity, HVAC control systems, or vapor deposition systems.

In any preceding embodiment, the polymeric film bearing the raised features and infused with the lubricant has channels within the film that carry lubricating liquid. The polymeric film has inside it or at the surface attached to the substrate an artificial self-replenishing vascularized network of channels that provide a reservoir of the liquid transported inside the film.

In another aspect, the device as described herein is used as thermal power plant condensers, water harvesters, desalination plants, distillation towers (e.g., hydrocarbons, polyolefins, hydrofluorocarbons), building thermal/humidity control systems, or vapor deposition systems.

In another aspect, a method of condensing a phase change material on surface is provided including providing a phase-change device as described herein, and exposing the heat exchanger to a form of a phase change material wherein the phase change material undergoes a phase change and heat is released or absorbed.

In any of the preceding embodiments, the phase change material condenses as a droplet on the device.

In any of the preceding embodiments, the droplets are directionally guided to shed in a predetermined direction.

In any of the preceding embodiments, the droplets are directionally guided to shed along the direction determined by the widening slope of the asymmetric feature.

In any of the preceding embodiments, shedding direction is not determined by the orientation of the bump relative to gravity.

In any of the preceding embodiments, the a thermally conductive substrate is in the form of a pipe or coil.

In any of the preceding embodiments, the phase change material is water, polyolefins, hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, bromochlorofluorocarbons, perfluorocarbons, hydrofluorocarbons, hydrofluoroolefins, brines, eurammon, azeotropic compound, and refrigerants.

In any of the preceding embodiments, the phase change material is a refrigerant and the material can be one or more of 1,1,1,2,2,3,3,4,4-Nonafluorobutane, Carbon tetrachloride (Tetrachloromethane), Trichlorofluoromethane, Dichlorodifluoromethane, Bromochlorodifluoromethane, Dibromodifluoromethane, Chlorotrifluoromethane, Bromotrifluoromethane, Tetrafluoromethane, Chloroform (Trichloromethane), Dichlorofluoromethane, Chlorodifluoromethane, Bromodifluoromethane, Trifluoromethane (Fluoroform), Dichloromethane (Methylene chloride), Chlorofluoromethane, Difluoromethane, Chloromethane, Fluoromethane, Methane, Hexachloroethane, Pentachlorofluoroethane, 1,1,2,2-Tetrachloro-1,2-difluoroethane, 1,1,1,2-Tetrachloro-2,2-difluoroethane, 1,1,2-Trichlorotrifluoroethane, 1,1,2-Trichlorotrifluoroethane, 1,2-Dichlorotetrafluoroethane, 1,1-Dichlorotetrafluoroethane, 1,2-Dibromotetrafluoroethane, Chloropentafluoroethane, Hexafluoroethane, Pentachloroethane, 1,1,2,2-Tetrachloro-1-fluoroethane, 1,1,1,2-Tetrachloro-2-fluoroethane, 1,1,2-Trichloro-2,2-difluoroethane, 1,1,2-Trichloro-1,2-difluoroethane, 1,1,1-Trichloro-2,2-difluoroethane, 2,2-Dichloro-1,1,1-trifluoroethane, 1,2-Dichloro-1,1,2-trifluoroethane, 1,1-Dichloro-1,2,2-trifluoroethane, 2-Chloro-1,1,1,2-tetrafluoroethane, 1-Chloro-1,1,2,2-tetrafluoroethane, Pentafluoroethane, Pentafluorodimethyl ether, 1,1,2,2-Tetrachloroethane, 1,1,1,2-Tetrachloroethane, 1,1,2-Trichloro-2-fluoroethane, 1,1,2-Trichloro-1-fluoroethane, 1,1,1-Trichloro-2-fluoroethane, Dichlorodifluoroethane, 1,1-Dichloro-2,2-difluoroethane, 1,2-Dichloro-1,1-difluoroethane, 1,1-Dichloro-1,2-difluoroethane, 1,2-Dibromo-1,1-difluoroethane, 1-Chloro-1,2,2-Trifluoroethane, 1-Chloro-2,2,2-Trifluoroethane, 1-Chloro-1,1,2-Trifluoroethane, 1,1,2,2-Tetrafluoroethane, 1,1,1,2-Tetrafluoroethane, Bis(difluoromethyl)ether, 1,1,2-Trichloroethane, 1,1,1-Trichloroethane (Methyl chloroform), 1,2-Dichloro-1-fluoroethane, 1,2-Dibromo-1-fluoroethane, 1,1-Dichloro-2-fluoroethane, 1,1-Dichloro-1-fluoroethane, Chlorodifluoroethane, 1-Chloro-1,2-difluoroethane, 1-Chloro-1,1-difluoroethane, 1,1,2-Trifluoroethane, 1,1,1-Trifluoroethane, Methyl trifluoromethyl ether, 2,2,2-Trifluoroethyl methyl ether, 1,2-Dichloroethane 1,1-Dichloroethane Chlorofluoroethane, 1-Chloro-1-fluoroethane 1,2-Difluoroethane, 1,1-Difluoroethane Chloroethane (ethyl chloride) Fluoroethane Ethane Dimethyl ether, 1,1,1,2,2,3,3-Heptachloro-3-fluoropropane, Hexachlorodifluoropropane 1,1,1,3,3-Pentachloro-2,2,3-trifluoropropane, 1,2,2,3-Tetrachloro-1,1,3,3-tetrafluoropropane, 1,1,1-Trichloro-2,2,3,3,3-pentafluoropropane, 1,2-Dichloro-1,1,2,3,3,3-hexafluoropropane, 1,3-Dichloro-1,1,2,2,3,3-hexafluoropropane, 1-Chloro-1,1,2,2,3,3,3-heptafluoropropane, 2-Chloro-1,1,1,2,3,3,3-heptafluoropropane, Octafluoropropane , 1,1,1,2,2,3-Hexachloro-3-fluoropropane, Pentachlorodifluoropropane, 1,1,1,3,3-Pentachloro-2,2-difluoropropane, Tetrachlorotrifluoropropane, 1,1,3,3-Tetrachloro-1,2,2-trifluoropropane, 1,1,1,3-Tetrachloro-2,2,3-trifluoropropane, Trichlorotetrafluoropropane, 1,3,3-Trichloro-1,1,2,2-tetrafluoropropane, 1,1,3-Trichloro-1,2,2,3-tetrafluoropropane, 1,1,1-Trichloro-2,2,3,3-Dichloropentafluoropropane, 2,2-Dichloro-1,1,1,3,3-pentafluoropropane, 2,3-Dichloro-1,1,1,2,3-pentafluoropropane, 1,2-Dichloro-1,1,2,3,3-pentafluoropropane, 3,3-Dichloro-1,1,1,2,2-pentafluoropropane, 1,3-Dichloro-1,1,2,2,3-pentafluoropropane, 1,1-Dichloro-1,2,2,3,3-pentafluoropropane, 1,2-Dichloro-1,1,3,3,3-pentafluoropropane, 1,3-Dichloro-1,1,2,3,3-pentafluoropropane, 1,1-Dichloro-1,2,3,3,3-pentafluoropropane, Chlorohexafluoropropane, 2-Chloro-1,1,1,2,3,3-hexafluoropropane, 3-Chloro-1,1,1,2,2,3-hexafluoropropane, 1-Chloro-1,1,2,2,3,3-hexafluoropropane, 2-Chloro-1,1,1,3,3,3-hexafluoropropane, 1-Chloro-1,1,2,3,3,3-hexafluoropropane, 1,1,2,2,3,3,3-Heptafluoropropane, Trifluoromethyl 1,1,2,2-tetrafluoroethyl ether, 1,1,1,2,3,3,3-Heptafluoropropane, Trifluoromethyl 1,2,2,2-tetrafluoroethyl ether, Pentachlorofluoropropane, Tetrachlorodifluoropropane, 1,1,3,3-Tetrachloro-2,2-difluoropropane, 1,1,1,3-Tetrachloro-2,2-difluoropropane, Trichlorotrifluoropropane, 1,1,3-Trichloro-2,2,3-trifluoropropane, 1,1,3-Trichloro-1,2,2-trifluoropropane, 1,1,1-Trichloro-2,2,3-trifluoropropane, Dichlorotetrafluoropropane, 2,2-Dichloro-1,1,3,3-tetrafluoropropane, ,2-Dichloro-1,1,1,3-tetrafluoropropane, 1,2-Dichloro-1,2,3,3-tetrafluoropropane, 2,3-Dichloro-1,1,1,2-tetrafluoropropane, 1,2-Dichloro-1,1,2,3-tetrafluoropropane-3-Dichloro-1,2,2,3-tetrafluoropropane, 1,1-Dichloro-2,2,3,3-tetrafluoropropane, 1,3-Dichloro-1,1,2,2-tetrafluoropropane, 1,1-Dichloro-1,2,2,3-tetrafluoropropane, 2,3-Dichloro-1,1,1,3-tetrafluoropropane, 1,3-Dichloro-1,1,3,3-tetrafluoropropane-1-Dichloro-1,3,3,3-tetrafluoropropane, Chloropentafluoropropane, 1-Chloro-1,2,2,3,3-pentafluoropropane, 3-Chloro-1,1,1,2,3-pentafluoropropane, 1-Chloro-1,1,2,2,3-pentafluoropropane, 2-Chloro-1,1,1,3,3-pentafluoropropane, 1-Chloro-1,1,3,3,3-pentafluoropropane, 1,1,1,2,2,3-Hexafluoropropane, 1,1,1,2,3,3-Hexafluoropropane, 1,1,1,3,3,3-Hexafluoropropane, 1,2,2,2-Tetrafluoroethyl difluoromethyl ether, Hexafluoropropane, Tetrachlorofluoropropane, Trichlorodifluoropropane, ichlorotrifluoropropane, 1,3-Dichloro-1,2,2-trifluoropropane, 1,1-Dichloro-2,2,3-trifluoropropane, 1,1-Dichloro-1,2,2-trifluoropropane, 2,3-Dichloro-1,1,1-trifluoropropane, 1,3-Dichloro-1,2,3-trifluoropropane, 1,3-Dichloro-1,1,2-trifluoropropane, Chlorotetrafluoropropane, 2-Chloro-1,2,3,3-tetrafluoropropane, 2-Chloro-1,1,1,2-tetrafluoropropane, 3-Chloro-1,1,2,2-tetrafluoropropane, 1-Chloro-1,2,2,3-tetrafluoropropane, 1-Chloro-1,1,2,2-tetrafluoropropane, 2-Chloro-1,1,3,3-tetrafluoropropane, 2-Chloro-1,1,1,3-tetrafluoropropane, 3-Chloro-1,1,2,3-tetrafluoropropane, 3-Chloro-1,1,1,2-tetrafluoropropane, 1-Chloro-1,1,2,3-tetrafluoropropane, 3-Chloro-1,1,1,3-tetrafluoropropane, 1-Chloro-1,1,3,3-tetrafluoropropane, 1,1,2,2,3-Pentafluoropropane, Pentafluoropropane, 1,1,2,3,3-Pentafluoropropane, 1,1,1,2,3-Pentafluoropropane, 1,1,1,3,3-Pentafluoropropane, Methyl pentafluoroethyl ether, Difluoromethyl 2,2,2-trifluoroethyl ether, Difluoromethyl 1,1,2-trifluoroethyl ether, Trichlorofluoropropane, Dichlorodifluoropropane, 1,3-Dichloro-2,2-difluoropropane, 1,1-Dichloro-2,2-difluoropropane, 1,2-Dichloro-1,1-difluoropropane, 1,1-Dichloro-1,2-difluoropropane, Chlorotrifluoropropane, 2-Chloro-1,2,3-trifluoropropane, 2-Chloro-1,1,2-trifluoropropane, 1-Chloro-2,2,3-trifluoropropane, 1-Chloro-1,2,2-trifluoropropane, 3-Chloro-1,1,2-trifluoropropane, 1-Chloro-1,2,3-trifluoropropane, 1-Chloro-1,1,2-trifluoropropane, 3-Chloro-1,3,3-trifluoropropane, 3-Chloro-1,1,1-trifluoropropane, 1-Chloro-1,1,3-trifluoropropane, 1,1,2,2-Tetrafluoropropane, ethyl 1,1,2,2-tetrafluoroethyl ether, Dichlorofluoropropane, 1,2-Dichloro-2-fluoropropane, Chlorodifluoropropane, 1-Chloro-2,2-difluoropropane, 3-Chloro-1,1-difluoropropane , 1-Chloro-1,3-difluoropropane, Trifluoropropane , Chlorofluoropropane, 2-Chloro-2-fluoropropane, 2-Chloro-1-fluoropropane, 1-Chloro-1-fluoropropane, Difluoropropane, Fluoropropane, Propane, Dichlorohexafluorocyclobutane , Chloroheptafluorocyclobutane, Octafluorocyclobutane, (Perfluorocyclobutane), Decafluorobutane (Perfluorobutane), 1,1,1,2,2,3,3,4,4-Nonafluorobutane, 1,1,1,2,3,4,4,4-Octafluorobutane, 1,1,1,2,2,3,3-Heptafluorobutane, Perfluoropropyl methyl ether, Perfluoroisopropyl methyl ether, 1,1,1,3,3-Pentafluorobutane, Dodecafluoropentane (Perfluoropentane) , or Tetradecafluorohexane (Perfluorohexane).

In any of the preceding embodiments, the phase change material forms as a liquid (droplet), gas (bubble) or solid.

In any of the preceding embodiments, the solid is a particle in any shape or clusters of particles in any shape, or the phase change material is water, or the feature height is less than the depletion layer, or the features have a width on the dimension of the condensing droplets.

In any of the preceding embodiments, the asymmetric structures have a width that is the same as the diameter of the shedding droplets

In any of the preceding embodiments, the phase change device is a heat exchanger and further comprising transferring heat released or absorbed by the latent heat exchanger using a heat sink in thermal contact with the heat exchanger.

In any of the preceding embodiments, the heat exchanger is functional to effect condensation in multi-stage flash (MSF) desalination plants, thermal and humidity management systems for buildings, etc., liquid harvesting by facilitating the condensation of vapor, effective prevention of mechanical failure of underwater ship parts (e.g., motor screws) by the relief of impact of bubbles generated from cavitation, release of bubbles that hinder the transport of liquid in the pipe and release of inorganic and organic fouling.

In another aspect, a method of decoupling phase change material growth and transport includes providing a phase change-based device a deformable substrate comprising a plurality of macro-scale raised features having a convex surface, wherein the geometry of the feature promotes droplet, solid or bubble formation and accelerated growth on the apex of the raised feature; and condensing a phase change material on at last the apices of the macro-scale raised features or the device; and deforming the substrate and the macro-scale raised features to remove a phase of a phase-change material from the apices.

Convexity, or raised topography in a broader sense, can also contribute to faster droplet formation due to the focusing effect of diffusion flux of the incoming phase before phase change on the convex surface texture. In one or more embodiments, the SLIPS-based heat exchanger surfaces demonstrate “convexity effects” that nucleate large droplets and bubbles in shorter time scales compared to state-of-the-art superhydrophobic or traditional SLIPS-treated surfaces, and significantly shorter times compared to regular, untreated condenser materials. In one or more embodiments, a latent heat exchanger or condenser contains nanometer to centimeter scale convex surface structures that are treated with a SLIPS surface; the device shows several times faster droplet growth and shedding compared to superhydrophobic or traditional SLIPS-treated surfaces, and significantly faster times compared to regular, untreated condenser materials.

The individually described embodiments of the claim may be used in the alternative and in combination with any other embodiments described within.

These and other aspects and embodiments of the disclosure are illustrated and described below.

### BRIEF DESCRIPTION OF THE DRAWINGS

The invention is described with reference to the following figures, which are presented for the purpose of illustration only and are not intended to be limiting.

In the Drawings:

FIG. 1A-1B are schematic illustrations of exemplary convex structures that be used for droplet, bubble or particle nucleation and growth according to one or more embodiments; FIG. 1A(i)-(iii) illustrates a series of surfaces including (i) symmetrical convex features, (ii) asymmetrical convex features containing a single slope for directional transport and (iii) asymmetric and symmetric convex features containing slopes in multiple directions for multidirectional transport; and FIG. 1B(i)-(iii) illustrates similar features that further contain a SLIPS coating.

FIG. 2A is an optical profilometer image of an exemplary convex structure that can be used for the SLIPS-based heat exchanger surfaces, according to one or more embodiments.

FIG. 2B shows SEM (top) and contact profilometer (bottom) images of spherical-cap-shaped bumps without PDMS coating (i.e., aluminum, left images) as well as SEM and contact profilometer images of spherical-cap-shaped bumps with PDMS coating (right images)

FIG. 2C shows a profilometer image of a hexagonal array of millimeter-scale spherical-cap-shaped bumps (left), and the location of the plane below which diffusion is the dominant mechanism of mass transport (depletion layer δ>>H, dotted line, right).

FIG. 2D shows time-lapsed images of droplets growing by condensation on the apex of the bumps (top row) compared to a flat region with the same height H (bottom row). The largest droplet in each series is denoted by dotted circles at each time point.

FIG. 2E shows that (left) the spherical-cap-shaped bump without additional roughening by sandpaper exhibits larger droplets on its apex compared to those of (right) the roughened flat surfaces of the square-shaped bump with the same height, thus ruling out the importance of the micro/nano surface roughness to the observed preferential droplet growth at the apex of the structures.

FIG. 2F shows three different schematic illustrations for deriving a simple scaling law to describe the effect of radius of curvature on diffusion flux (J_{C}) focused on the apex of spherical-cap-shaped bumps: (left) Schematic illustration (cross-sectional view) of depletion layer (represented by dotted line) and a spherical-cap-shaped bumped surface (represented by solid line), in which the rectangle and point represent the area and point of interest, respectively; (center) a spherical mass sink corresponding to the spherical-cap-shaped bump; and (right) the spherical mass sink and its mirror image (a corresponding spherical mass source represented by dotted line) used to calculate the concentration distribution and gradient near the point of interest.

FIG. 2G is a plot based on an analytical model that predicts magnitude of diffusion flux (J_{C}) on the apex of a spherical bump as a function of the radius of curvature (κ⁻¹). Values are normalized to diffusion flux on flat surfaces with the same H.

FIG. 2H shows time-dependent droplet growth on bumps with decreasing radii of curvature at the apex. 2rₘₐₓ is the averaged diameter of the three largest droplets found on the apex of the bumps.

FIGS. 3A-3B are illustrations of an edge-containing structure design according to one or more embodiments.

FIG. 3C is a schematic illustration of various raised features according to one or more embodiments.

FIG. 3D is an optical image of condensed droplets on rectangular bumps with the same height but different widths.

FIG. 3E shows time-dependent droplet growth on bumps with different width. Droplets on the rectangular bump with the smallest width can grow for a longer time at a constant growth rate, leading to a greater droplet size compared to spherical-cap-shaped bumps at later time points.

FIG. 4A shows droplet transport directions on a rectangular bump;

FIG. 4B shows droplet transportation a bump modified with an asymmetric slope.

FIG. 4C shows energy profile normalized by the difference between the maximum and minimum value of total free energy of the asymmetric bump-droplet-vapor system, obtained by finite element-based numerical calculation (Surface Evolver).

FIG. 4D is a profilometer image (oblique view) of a friction-free asymmetric bump according to one or more embodiments, showing nanostructure that is infused with lubricant to create a molecularly smooth slippery coating, and the tangentially connected bottom slope used to prevent pinning

FIG. 4E (i)-(iv) are schematic illustrations of various raised features containing a slope tangentially transitioning from the apex of the feature to the surface according to one or more embodiments.

FIG. 4F shows time-lapsed optical images of condensed water droplets on an asymmetric bump rotated 180° relative to gravity and showing that while on the bump, the droplet is moving against gravity in the direction determined by the orientation of the slope.

FIG. 4G shows time-lapsed optical images of condensed water droplets on an exemplary asymmetric structure in combination with the SLIPS coating rotated 90° relative to gravity showing that the droplet travels solely in the direction determined by the bump geometry until it reaches the end of the bump and makes an immediate 90° turn to align with gravity. The dotted line tracks the horizontal motion of the droplet on the bump.

FIG. 4H is a series of time-elapsed photographs of an exemplary asymmetric structure in combination with the SLIPS coating (i.e., SLIPS-A) (i)-(iii), showing more than 20-fold reduced time for the first shedding droplet compared to superhydrophobic surfaces (iv) and SHS-A (i.e., a superhydrophobic surface with asymmetric features) (v) due to the topographically-preferential condensation induced by a convex structure, forced coalescence and directional transport induced by the asymmetry of the design, and fast droplet movement induced by the negligible friction on SLIPS-A.

FIG. 41 shows quantitative analysis of growth dynamics and shedding for slippery asymmetric bumps (▴), superhydrophobic asymmetric bumps (Δ), flat slippery control (▪) and superhydrophobic (□) control. Each data point represents the averaged value of the largest droplet's growth on each of three different bumps or locations in the case of flat surfaces. Both slippery asymmetric bumps (▴) and superhydrophobic asymmetric bumps (Δ) show faster localized droplet growth in the early stage (t<10³ sec) compared to flat slippery (▪) and superhydrophobic (□) controls. Drops on slippery asymmetric bumps exhibit a further enhanced growth rate (rectangular box in the middle of the plot) as they move down the bump slope, approximately six-fold greater than typical droplet growth dynamics, and the shortest t_{first} (i.e., the average time at which the first three drops are transported solely by gravity) of <˜20 min. Each data point in the red box represents the averaged size of these three drops. Inset shows a magnified view of the fast growth, attributed to positive feedback between coalescence-driven growth and capillary transport. Neither superhydrophobic asymmetric bumps nor flat superhydrophobic surfaces produce shedding droplets within t˜200 min.

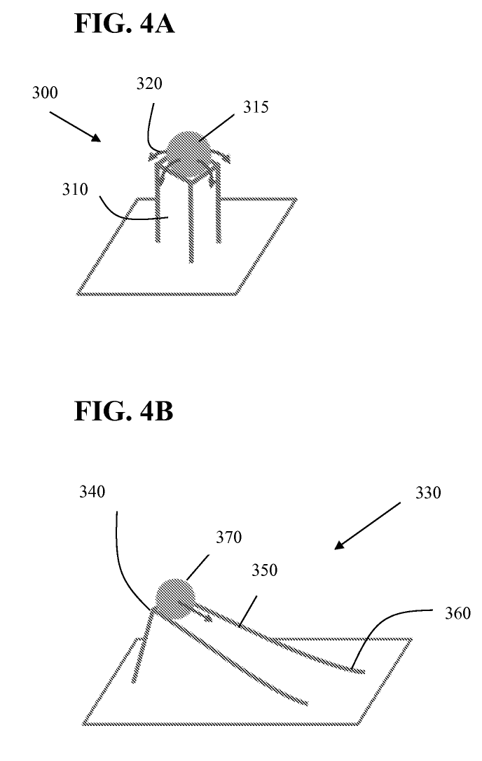

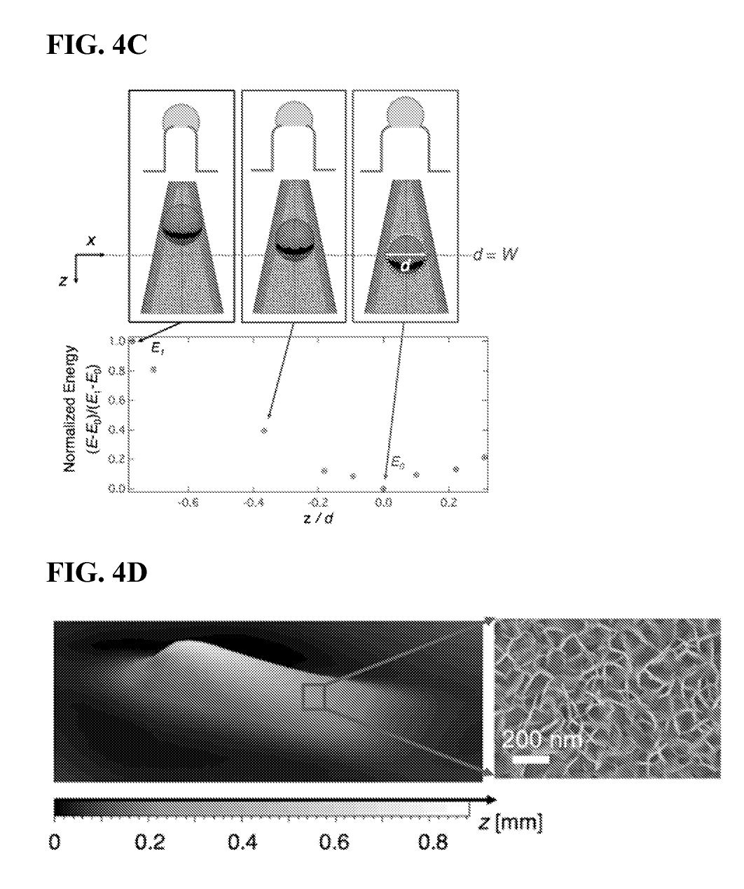

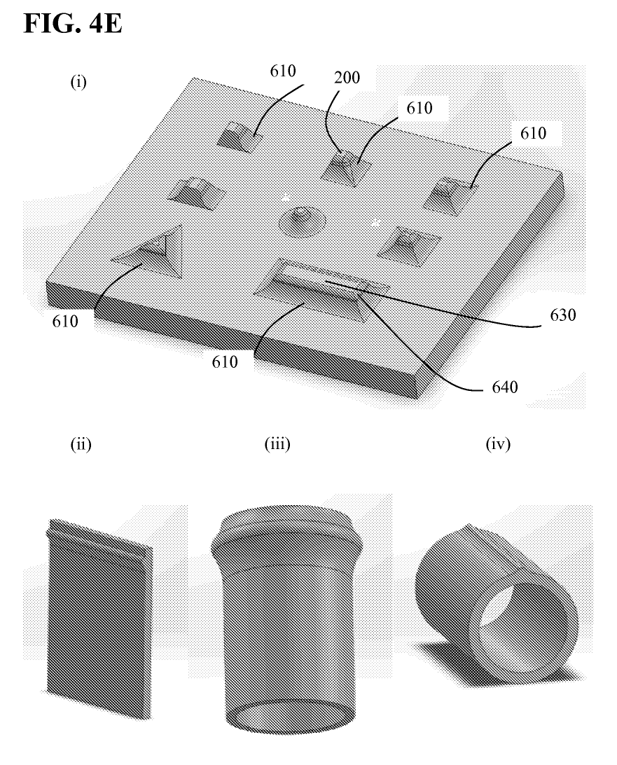

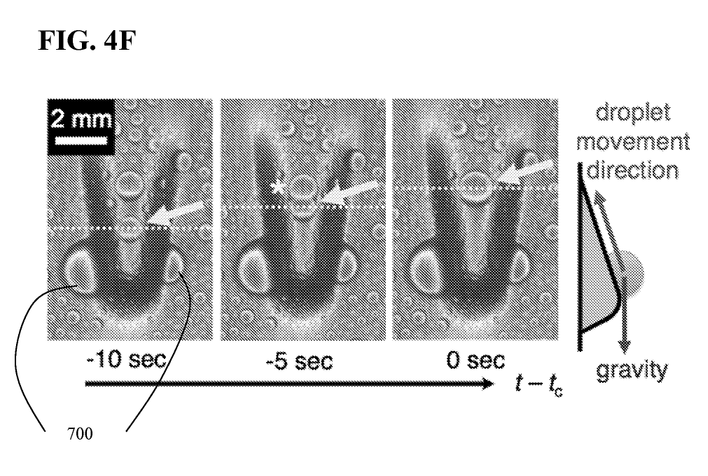

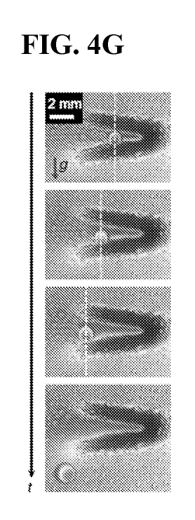

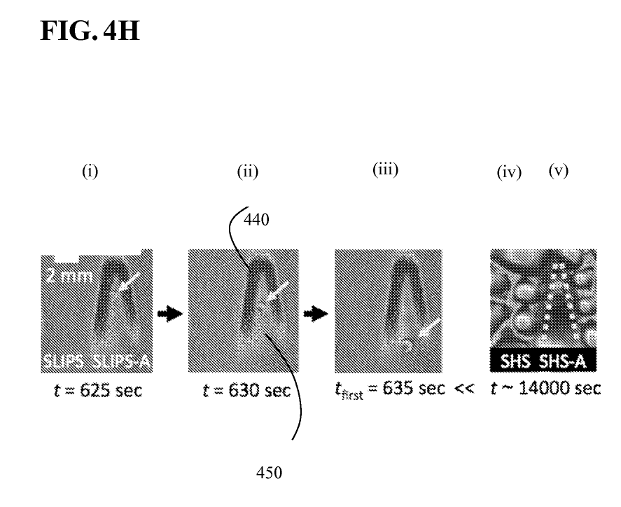

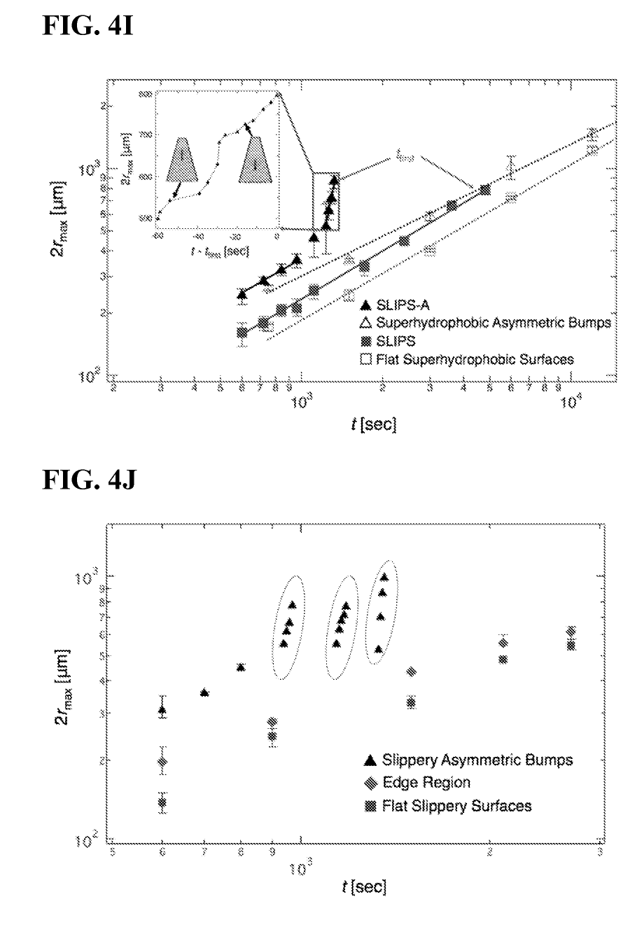

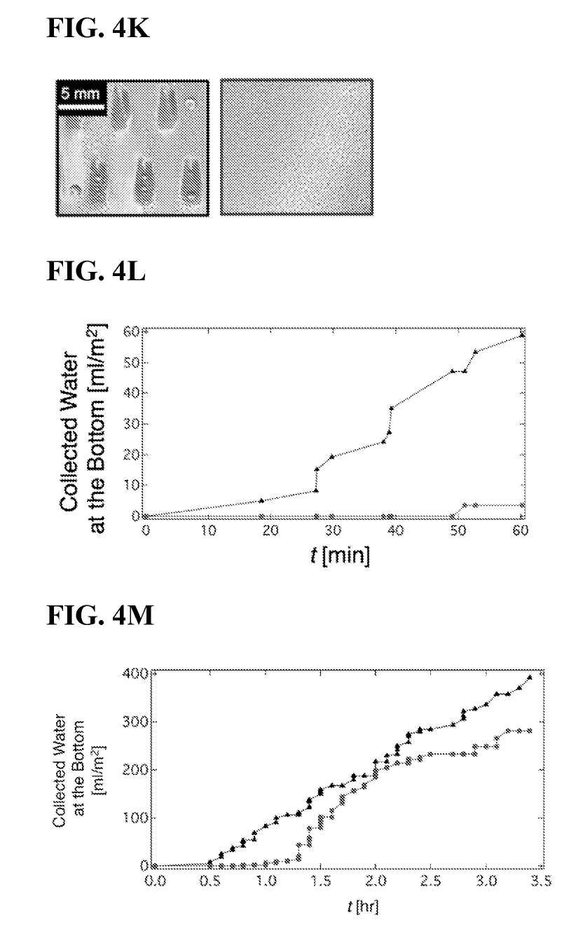

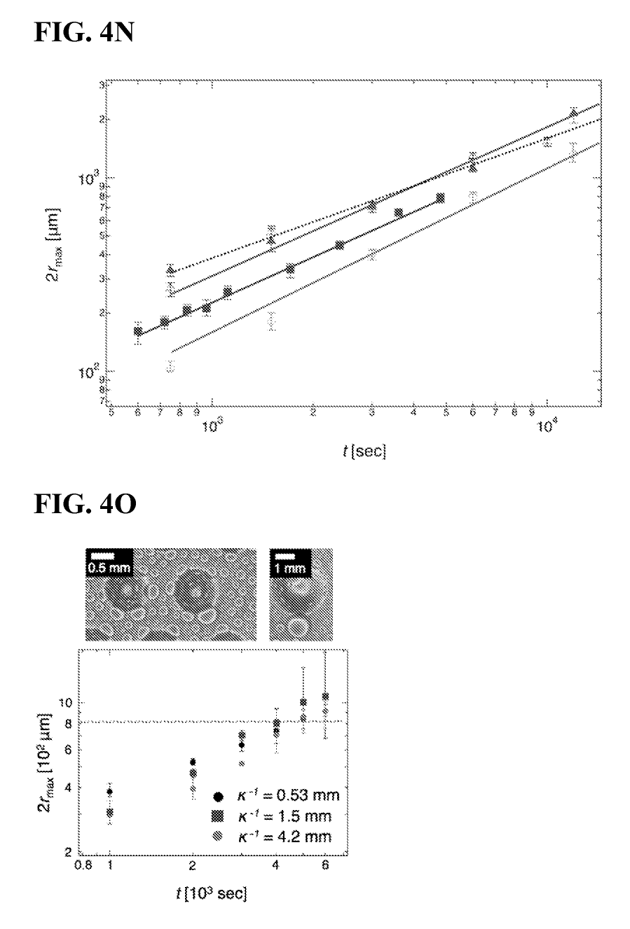

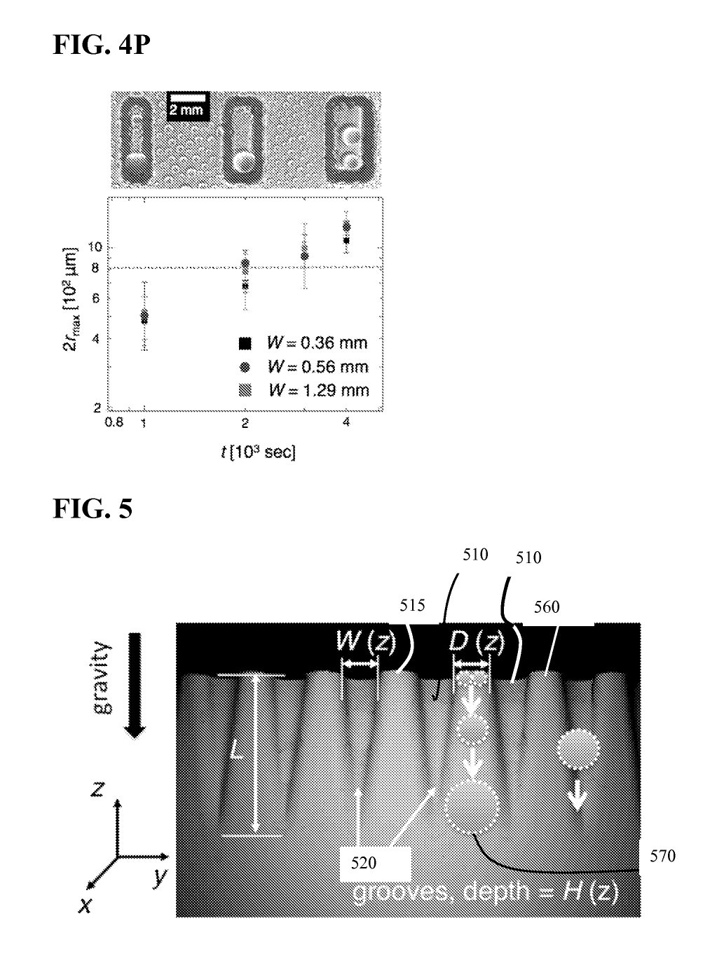

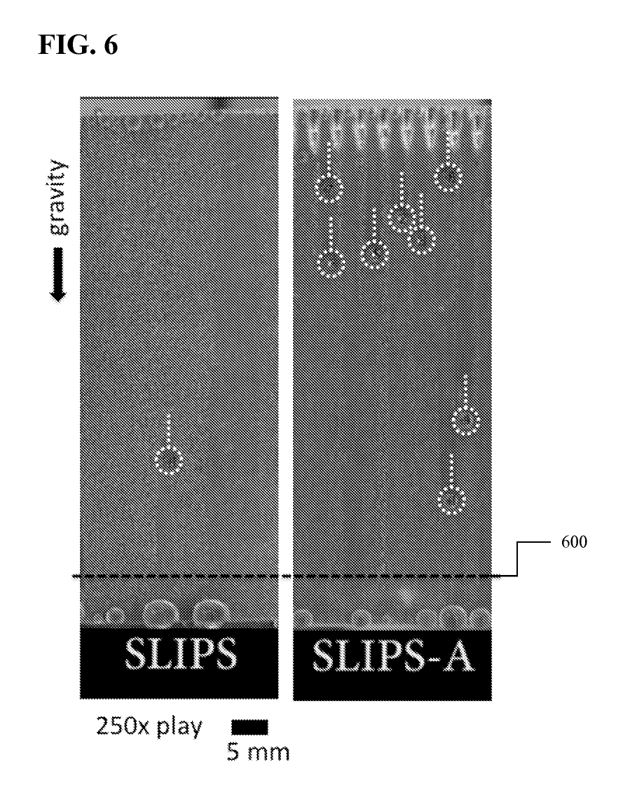

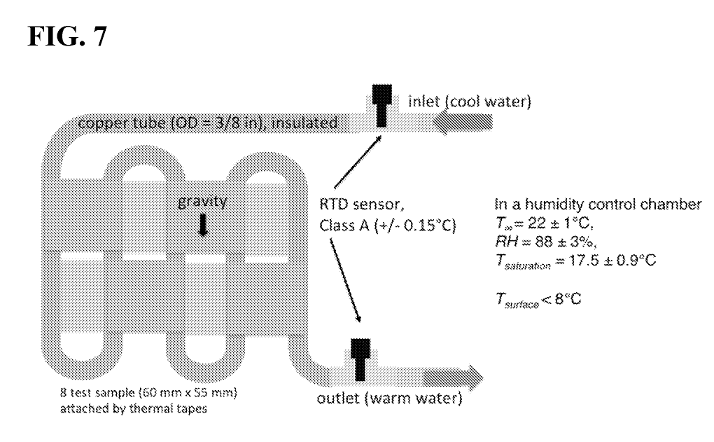

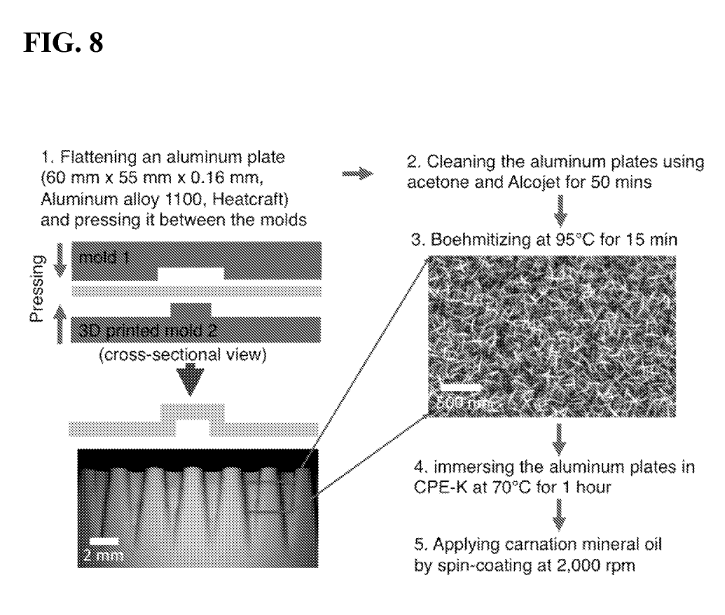

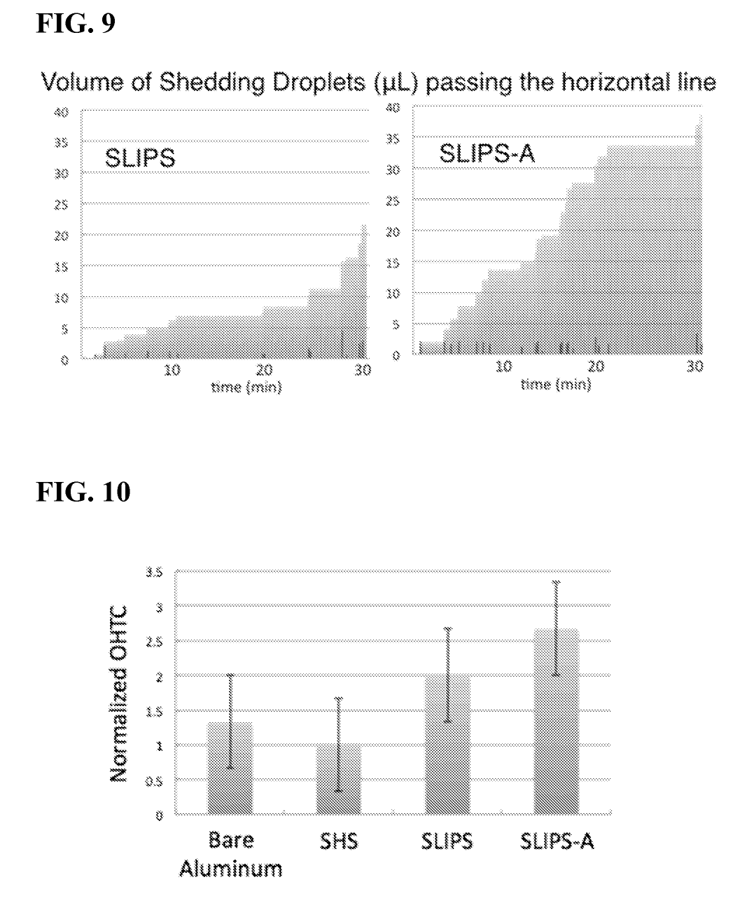

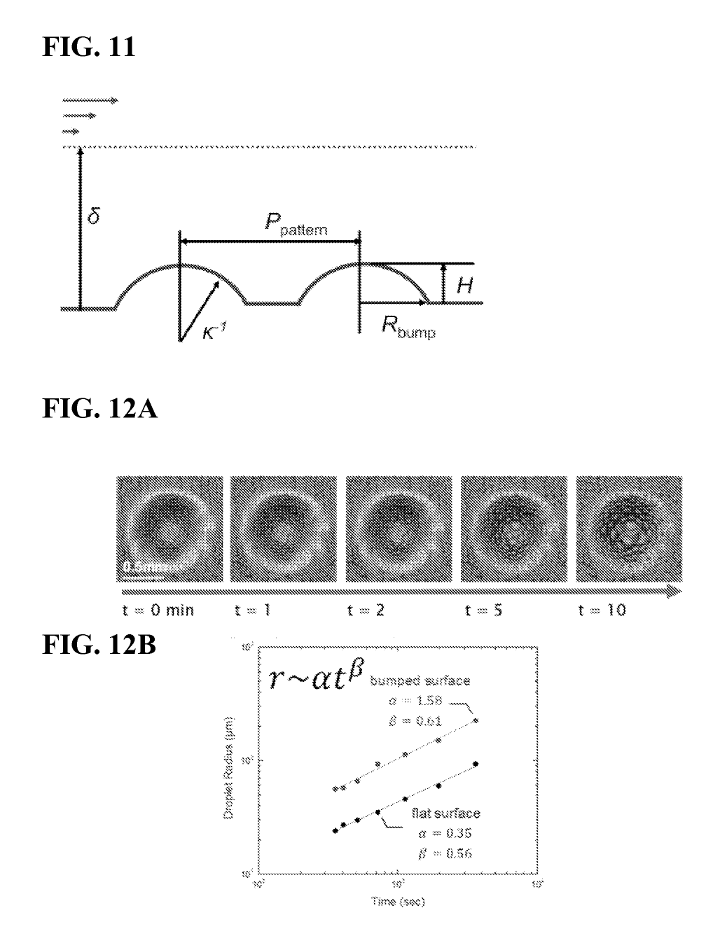

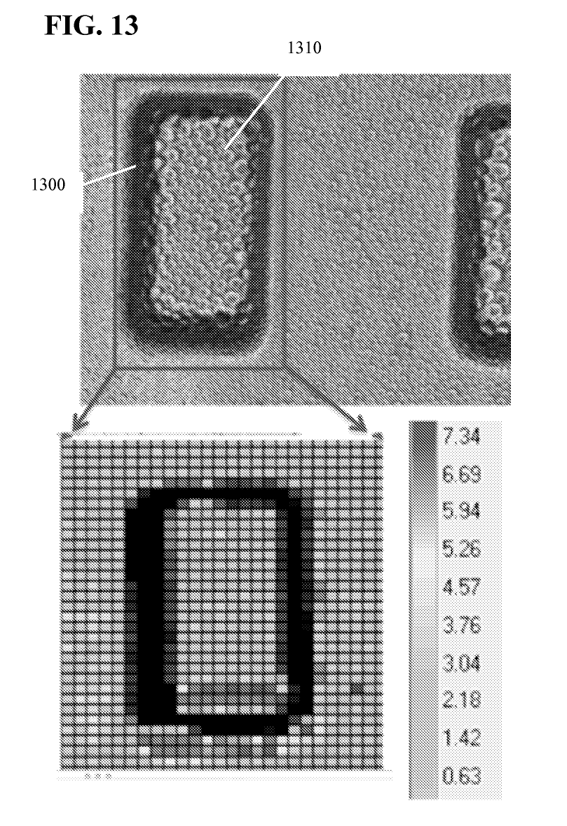

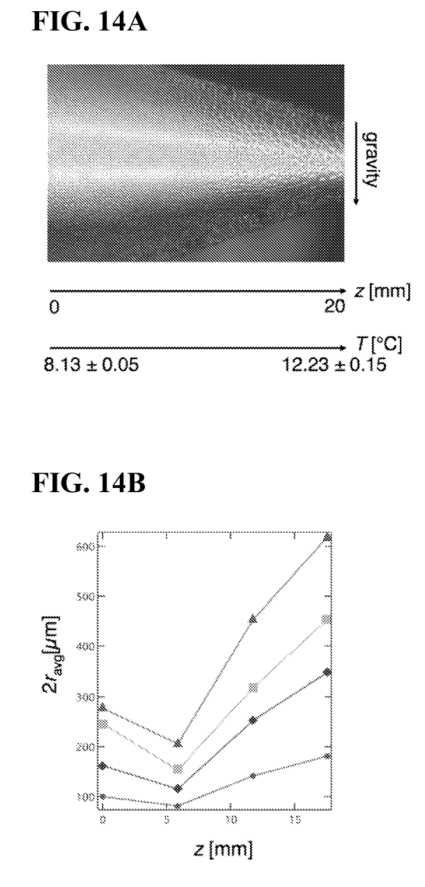

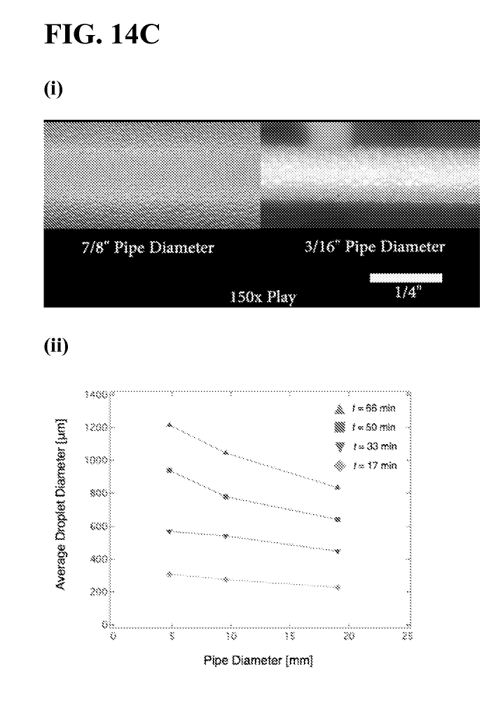

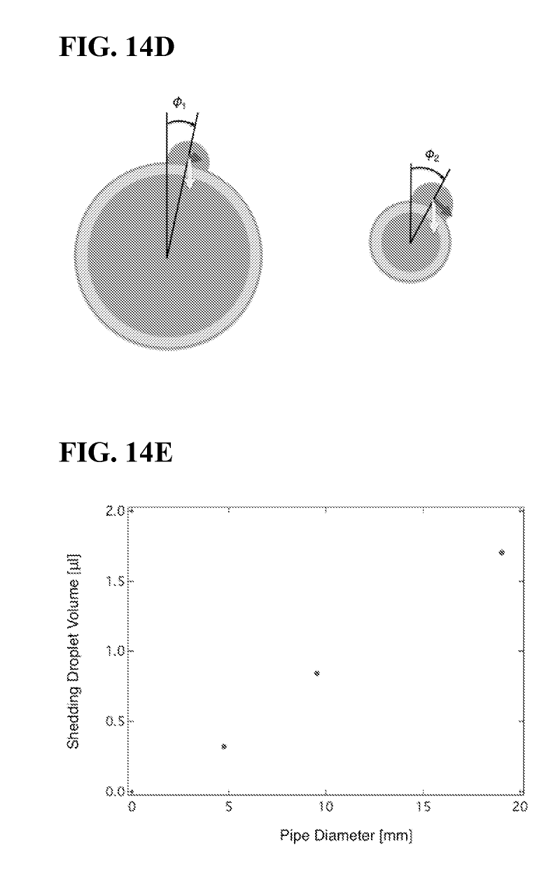

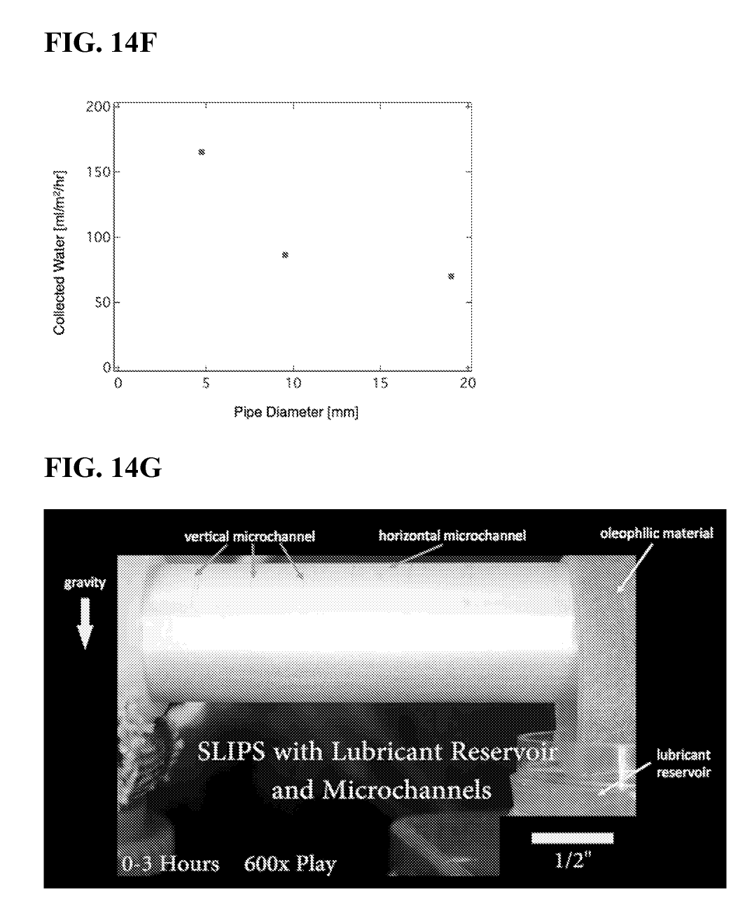

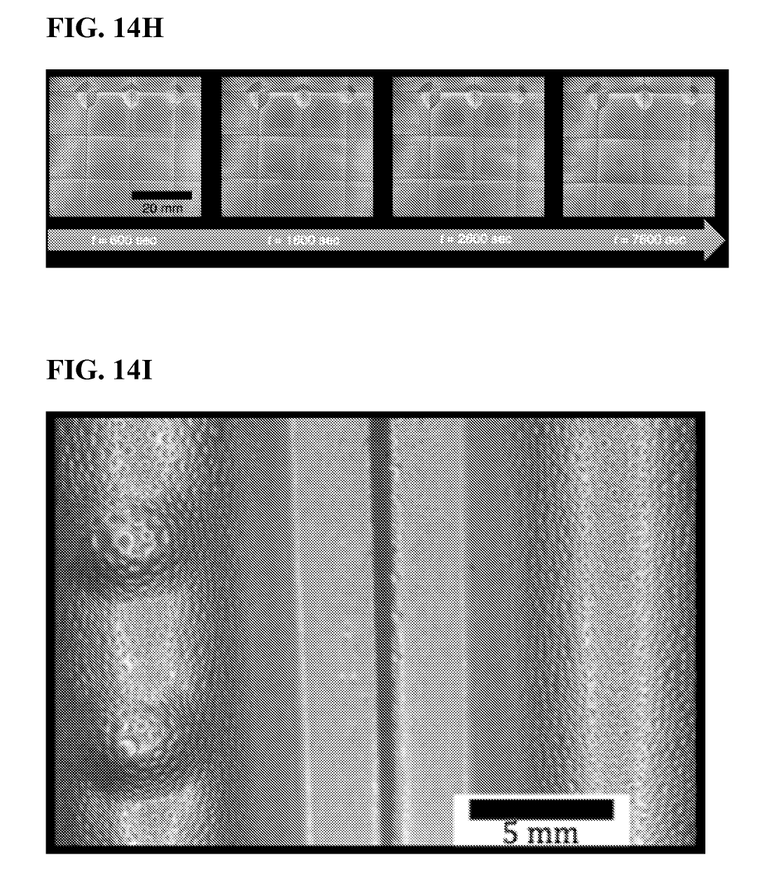

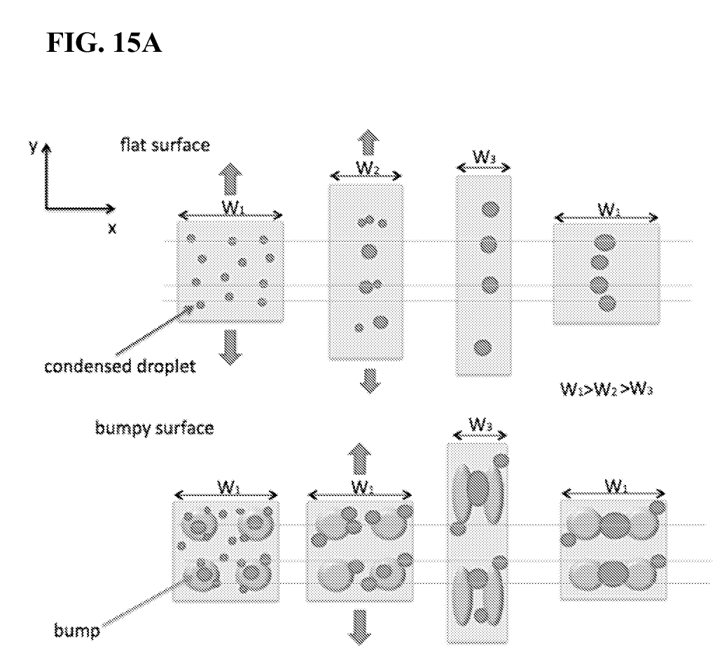

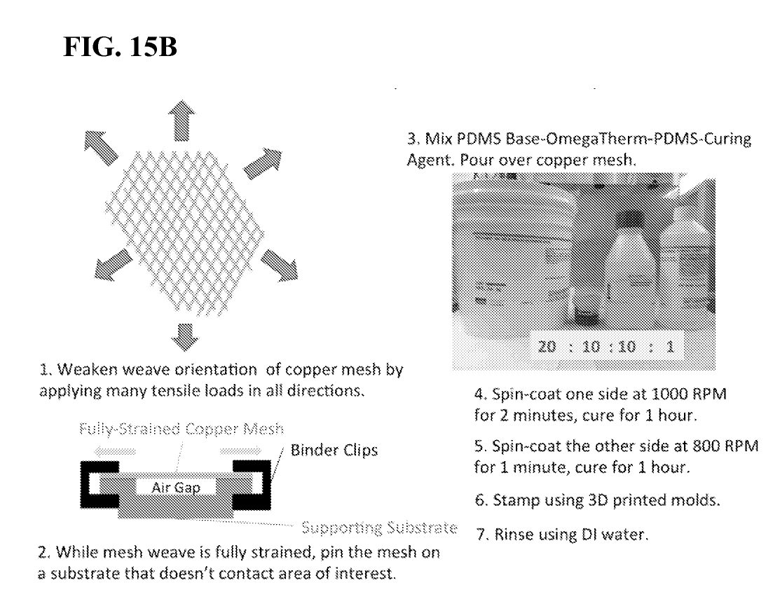

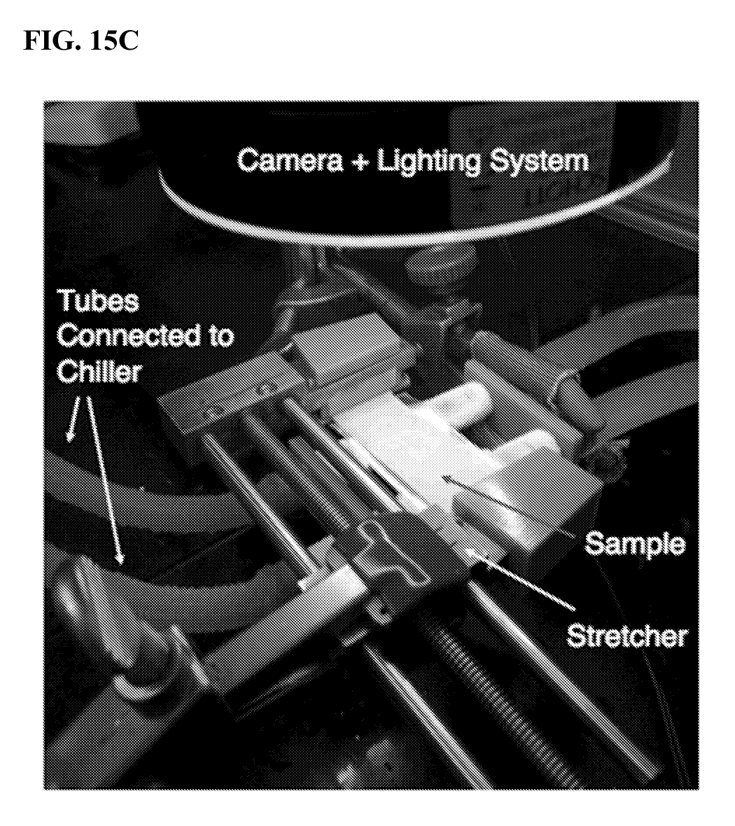

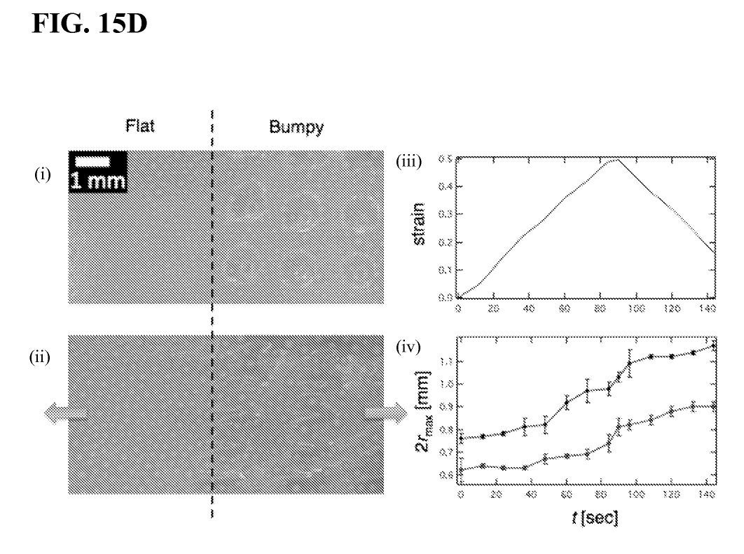

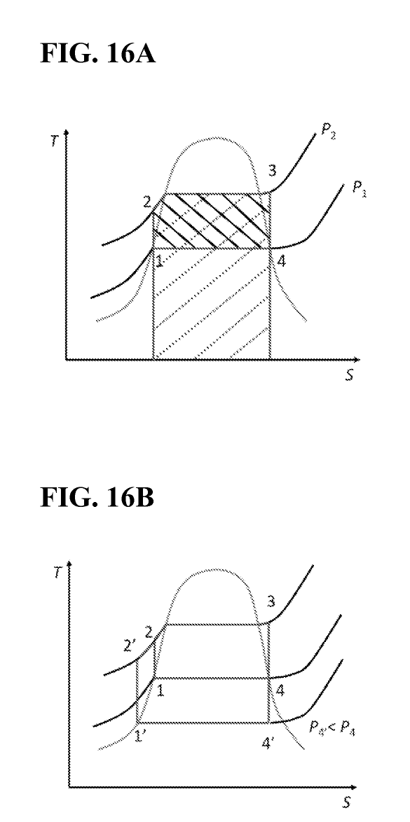

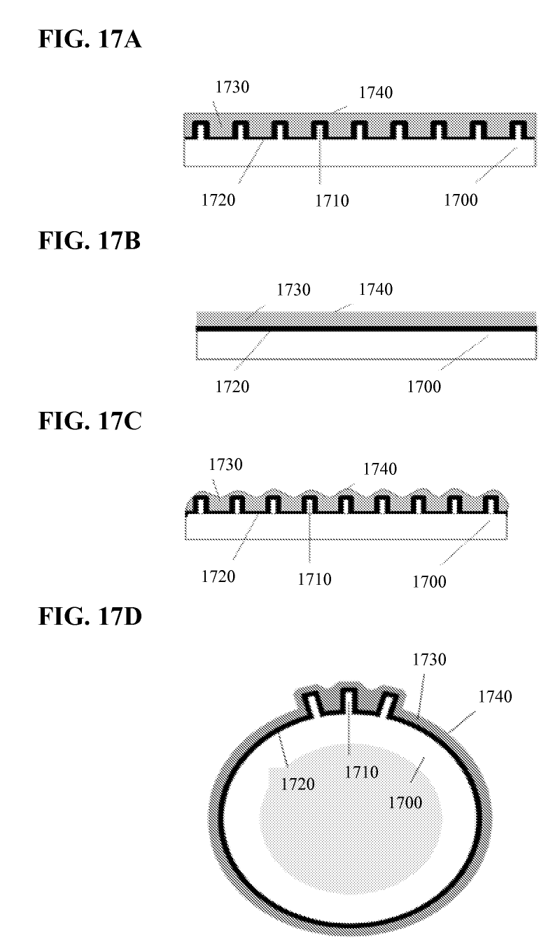

FIG. 4J is a plot of droplet diameter over time showing fast growth and transport of droplets on slippery asymmetric bumps compared to bottom edge regions. Each data point outside the three circles represents the average size of at least the three largest droplets on each surface. Each data point inside the three circles represents the diameter of the largest droplet on each of the bumps, obtained by tracking the same droplet on each of three different bumps.

FIG. 4K shows droplet condensation for an exemplary array of the slippery asymmetric bumps (left) compared to the flat slippery surfaces (right).

FIG. 4L shows a plot of collected water for the array of the slippery asymmetric bumps (▴) and flat slippery surfaces (▪), demonstrating that an exemplary array of the slippery asymmetric bumps (triangle in the plot) shows a significantly greater volume of water collected at the bottom of the surface, compared to the flat slippery surfaces (square in the plot).

FIG. 4M shows steady state water collection performance on bumpy (▴) and flat (▪) slippery surfaces.

FIG. 4N shows droplet growth curve on four representative types of surfaces—macroscopic asymmetric bumps with nanostructure (without lubricant, Δ, slope=0.62, SHS-A), macroscopic asymmetric bumps with molecularly smooth lubricant (▴, slope=0.78, SLIPS-A), flat slippery coatings (without macroscopic bump, ▪, slope=0.79, SLIPS), and flat superhydrophobic surfaces (as a control, ◯, slope=0.86, SHS).

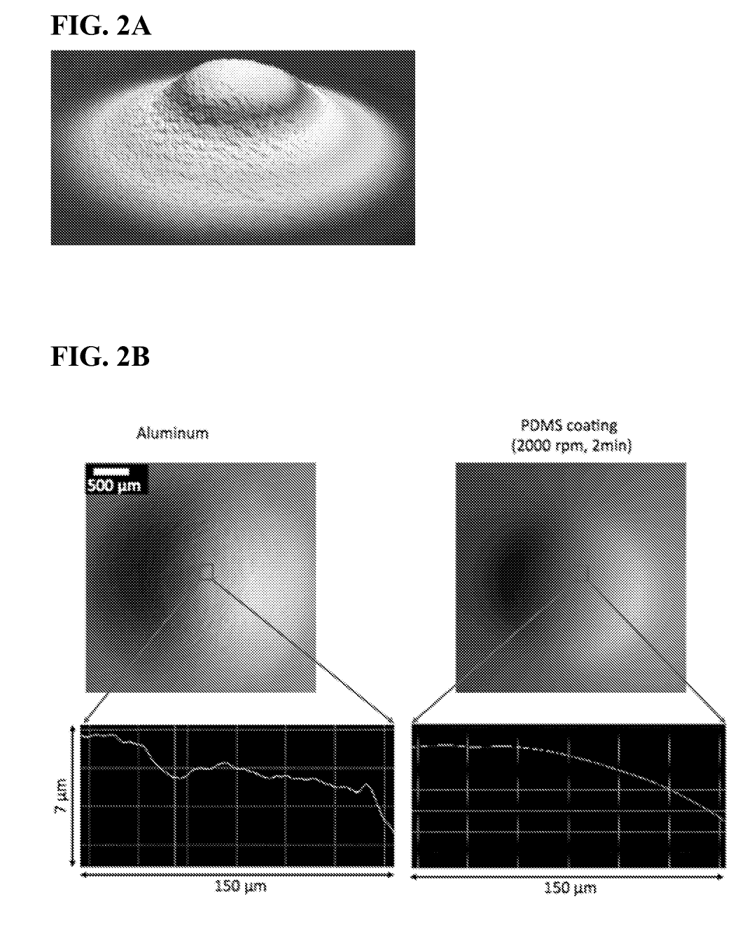

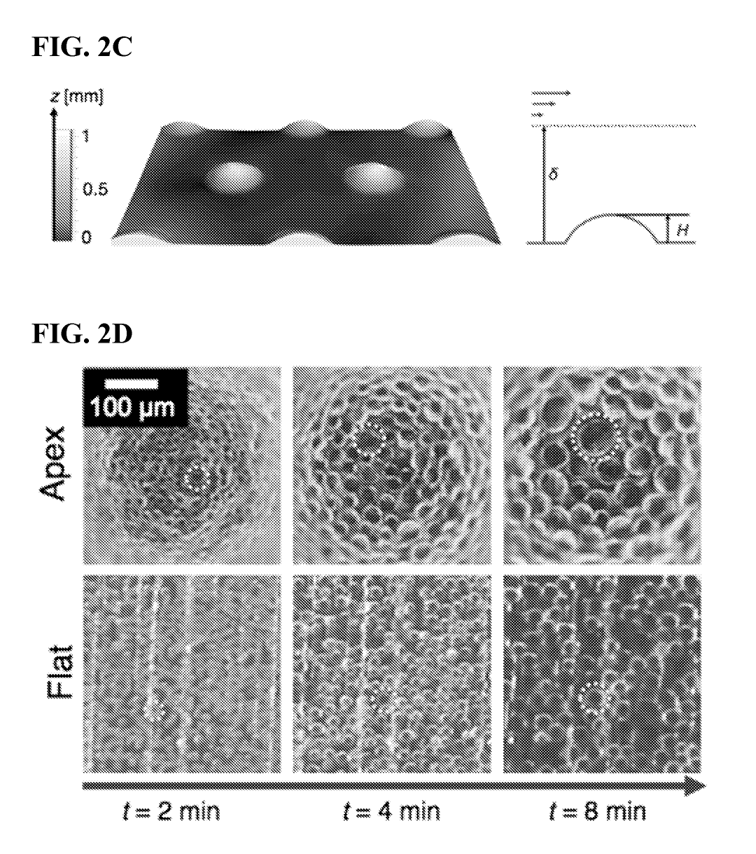

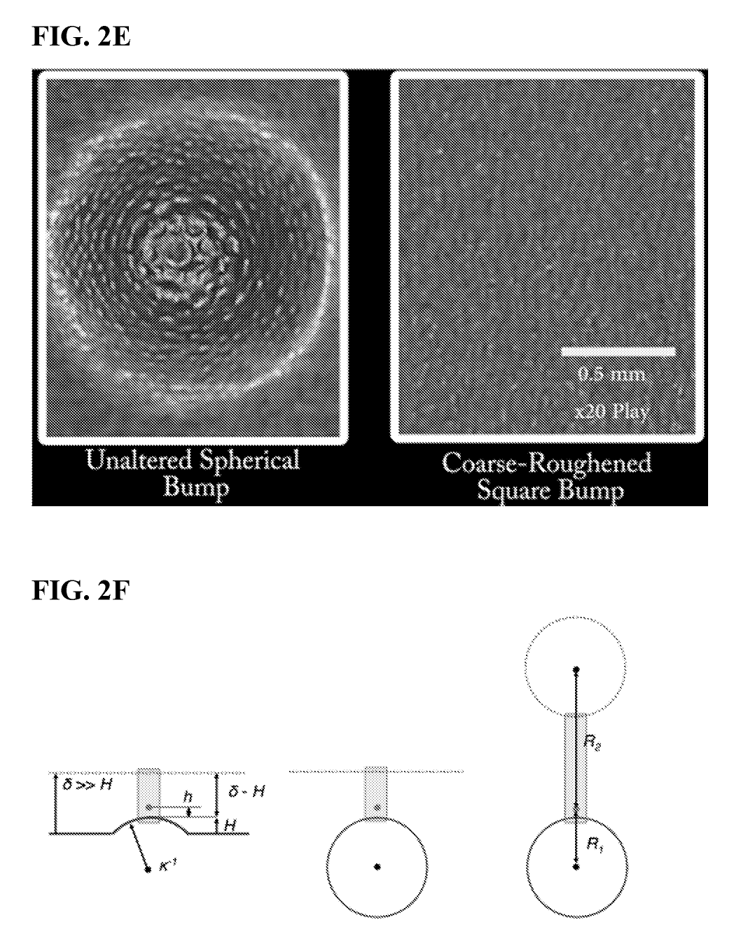

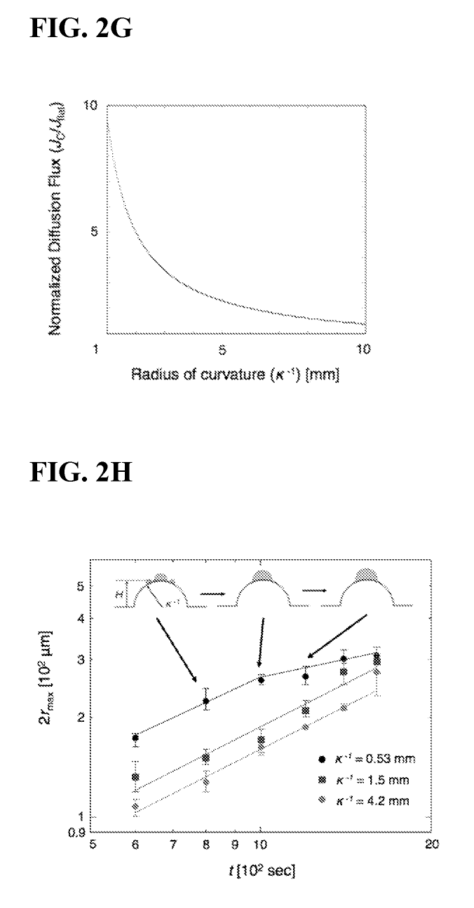

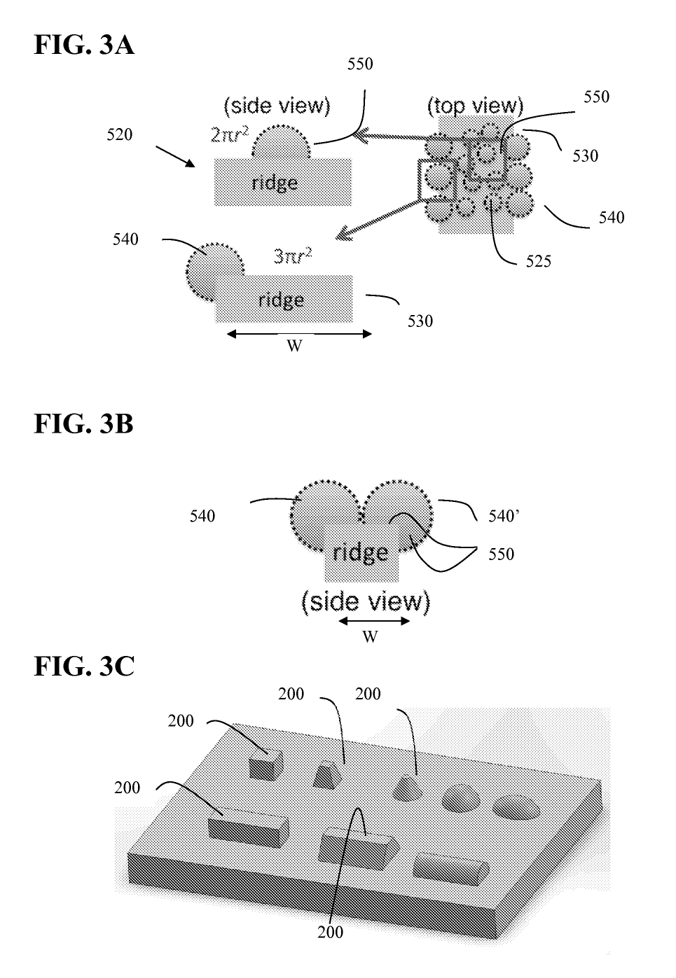

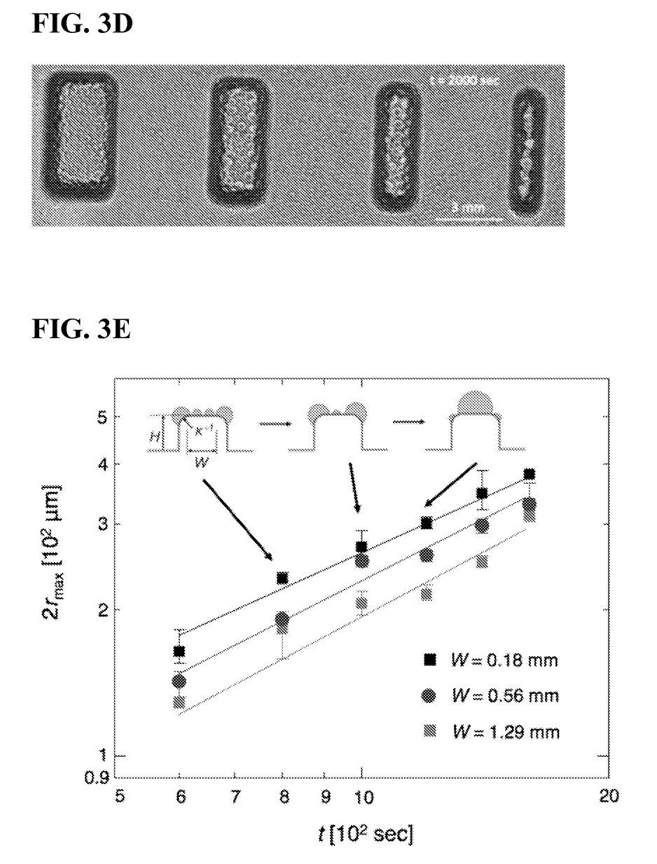

FIG. 4O and FIG. 4P show images of droplets condensed on slippery (FIG. 4O) spherical-cap-shaped bumps and (FIG. 4P) rectangular bumps. Even with slippery coatings, the bumps used in FIG. 2H and FIG. 3E did not display shedding droplets even though the droplets are greater than the shedding droplet diameter (denoted by dotted horizontal line) measured on flat surfaces, showing the importance of the asymmetric topography of bumps with the presence of a directional ramp.

FIG. 5 is an illustration of an exemplary asymmetric structure designs with geometrical parameters according to one or more embodiments.

FIG. 6 is an image showing the results of computational image analysis of experimental results of shedding droplets on different surfaces, in which the hollow white dotted circles represent the detected droplets and the tails with vertical white dotted lines represent the trace of shedding droplets. The time and diameter of the shedding droplets passing the horizontal black dotted line are recorded using MATLAB codes

FIG. 7 is a schematic illustration of the overall heat transfer coefficient measurement setup.

FIG. 8 is a schematic illustration of an exemplary high-throughput manufacturing process with the images of fabricated hierarchical asymmetric structures at different length scales.

FIG. 9 is a plot of the volume or the droplets shed as a function of time from SLIPS and SLIPS-A surfaces of FIG. 6.

FIG. 10 is a plot of overall heat transfer coefficient (OHTC) values normalized by the OHTC value of SHS (OHTC/OHTC_{SHS}) comparing bare aluminum surface, SHS surface, traditional SLIPS surface and hierarchical SLIPS surface with rationally designed convex and asymmetric features (SLIPS-A).

FIG. 11 is a schematic cross-sectional illustration of a raised feature according to one or more embodiments, illustrating surface feature parameters R_{bump} (radius of curvature of a convex hemispherical raised feature) Pₚₐₜₜₑᵣₙ (center to center distance between adjacent ‘bumps’), and H (height of a raised feature), as well as δ (depletion layer thickness below which mass transport (e.g., vapor) is governed by diffusion, according to one or more embodiments.

FIG. 12A and FIG. 12B are analyses of droplet growth dynamics on both the bumped and the surrounding flat surface, in which FIG. 12A is a time-lapsed sequential images of condensed droplets on the same bump and surrounding; and FIG. 12B is a quantitative analysis of droplet growth with time following the simple model (r˜αt^{β}).

FIG. 13 is an infrared camera image (bottom) of the rectangular bump (top, optical camera image), which shows no distinct temperature difference between the top flat area of the rectangular bump and the surrounding basal region. The unit of temperature next to the color bar is in ° C.

FIG. 14A is an image of water droplets condensed on a conical surface across which a temperature gradient was applied after t=4×10³ sec; while the left side is kept at a lower temperature than right side, and therefore higher condensation would be expected on that side, the curvature of the tube increases to the right and this effect dominates the temperature effect, resulting in larger droplets formed on the side with smaller pipe radius.

FIG. 14B shows a family of averaged droplet size (2r_{avg}) as a function of position. Circle, diamond, square, and triangle represent t=1×10³, 2×10³, 3×10³, and 4×10³ sec, respectively.

FIG. 14C is (i) an image of pipes of different diameters with an identical hydrophobic coating (FS-100), illustrating the effect of radius of curvature on droplet size and (ii) a plot of droplet diameter with pipe diameter, showing the effect of the radius of curvature (or diameter) of pipe geometry, another kind of convex curvature geometry widely used for industrial heat exchanger design, on drop growth. As confirmed in SLIPS-A experiments, the smaller the diameter of pipes, the faster the growth of the droplets.

FIG. 14D is a schematic illustration of a droplet on SLIPS pipe (cross sectional view) Of different diameters, showing the effect of diameter of pipes on the magnitude of driving force (gravitational force's component that is tangential to the pipe surface) of droplet shedding.

FIG. 14E is a plot of initial volume of shedding droplets (N=10), supporting the additional favorable effect of smaller pipe diameter on shedding droplet volume (or size) leading to earlier transport of droplets.

FIG. 14F is a plot of resultant water collection on different SLIPS pipes. The combined effects of fast drop growth and transport on a smaller diameter pipe results in a much greater amount of collected water per unit area of pipes.

FIG. 14G is an exemplary setup of simple lubricant-replenishment system by employing microchannel and oleophilic material.

FIG. 14H shows an enlarged exemplary combination of microchannels running vertically and horizontally in a surface used to replenish lubricant over time.

FIG. 14I is an image of a surface including (right) hemicylindrical raised features and (left) hemicylindrical features having periodic raised bumps to create a hierarchical structure according to one or more embodiments.

FIG. 15A is a schematic illustration of drop movement and coalescence when underlying stretchable substrate is elongated in y-direction for a flat surface and a bumpy surface.

FIG. 15B is a schematic illustration of the fabrication steps for creating a dynamic harvester.

FIG. 15C is an image of an exemplary dynamic harvester according to one or more embodiments.

FIG. 15D shows the results of the condensation experiments using a dynamic harvester. Optical images show the predicted fast drop growth by (unstretched) bumps (i) and the effect of stretching the substrate on further growth by guided coalescence (ii). The plot on the bottom right (iv) shows gradual drop growth in the process of stretching and relaxing represented by strain-time plot (iii).

FIG. 16A and B represent an ideal Rankine cycle. High temperature steam with high pressure generated from the boiler (Q_{H}, 2 or 2′→3) rotates the turbine (W_{T}, 3→4 or 4′). While rotating the turbine, the saturated steam expands resulting in lower temperature and pressure. It undergoes a phase change from vapor to liquid in the condenser (Q_{L}, 4→ or 4′→1′) by heat exchange with coolants. The condensed water is pumped (W_{P}, 1→2 or 1′→2′) to the boiler and then reused to make this thermodynamic cycle. The abscissa and ordinate are entropy and temperature, respectively.

FIGS. 17A-17E are schematic representations of various SLIPS surface, showing examples of surfaces with immobilized lubricant overlayer: A) nano/microtextured surfaces with the roughness factor R>1; B) largely flat surfaces that utilize the natural roughness of the material (R=1); C) nano/microtextured surfaces with the roughness factor R>1 with the lubricant conformally coating the nano/micro-asperities; D) nano/microtextured curved surfaces having a positive radius of curvature with the roughness factor R>1 with the lubricant conformally coating the nano/micro-asperities; and E) a nano/micro/millimeter scale flexible solid film layer that can hold and diffuse the lubricating layer.

### DETAILED DESCRIPTION

A simple, scalable, environment-friendly, and low-cost design and fabrication of SLIPS-based phase change-based devices (e.g., condensation, vaporization, sublimation, frosting, melting, freezing) is described. As one example of phase change-based devices, a latent heat exchanger allows energy to be released or absorbed by a body during a constant-temperature process. A typical example is a change of state such as the freezing or boiling of liquid (e.g., water, hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, silicones, bromochlorofluorocarbons, perfluorocarbons, hydrofluorocarbons, hydrofluoroolefins, brines, eurammon, azeotropic compound, and compounds of aforementioned refrigerants) or the condensation of vapor (e.g., water, polyolefins, hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, silicones, bromochlorofluorocarbons, perfluorocarbons, hydrofluorocarbons, hydrofluoroolefins, brines, eurammon, azeotropic compounds, and refrigerants). The heat exchanger surface can be applied on a wide range of metallic surfaces (or any thermal-conductive surface including polymeric surfaces) and can be used in a broad range of heat exchangers to enhance the heat transfer performance in the temperature range from superfreezing to superheating conditions (at which phase changes such as condensation and boiling occur). In illustrating the invention, water is used as a specific example of a system that can undergo condensation and heat exchange. Water is used for the purpose of illustration only and it is contemplated that one can employ the phase change and latent heat absorbers described herein for a range of phase-change fluids, for example to condense phase-change fluids from vapor phase to liquid phase. Exemplary phase-change fluids include water, polyolefins, hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, bromochlorofluorocarbons, hydrofluoroolefins, silicones, brines, eurammon, azeotropic compound, and compounds of aforementioned refrigerants, or hydrofluorocarbon (such as for example fluorinerts and vertrels), refrigerants such as Carbon tetrachloride (Tetrachloromethane), Trichlorofluoromethane, Dichlorodifluoromethane, Bromochlorodifluoromethane, Dibromodifluoromethane, Chlorotrifluoromethane, Bromotrifluoromethane, Tetrafluoromethane, Chloroform (Trichloromethane), Dichlorofluoromethane, Chlorodifluoromethane, Bromodifluoromethane, Trifluoromethane (Fluoroform), Dichloromethane (Methylene chloride), Chlorofluoromethane, Difluoromethane, Chloromethane, Fluoromethane, Methane, Hexachloroethane, Pentachlorofluoroethane, 1,1,2,2-Tetrachloro-1,2-difluoroethane, 1,1,1,2-Tetrachloro-2,2-difluoroethane, 1,1,2-Trichlorotrifluoroethane, 1,1,2-Trichlorotrifluoroethane, 1,2-Dichlorotetrafluoroethane, 1,1-Dichlorotetrafluoroethane, 1,2-Dibromotetrafluoroethane, Chloropentafluoroethane, Hexafluoroethane, Pentachloroethane, 1,1,2,2-Tetrachloro-1-fluoroethane, 1,1,1,2-Tetrachloro-2-fluoroethane, 1,1,2-Trichloro-2,2-difluoroethane, 1,1,2-Trichloro-1,2-difluoroethane, 1,1,1-Trichloro-2,2-difluoroethane, 2,2-Dichloro-1,1,1-trifluoroethane, 1,2-Dichloro-1,1,2-trifluoroethane, 1,1-Dichloro-1,2,2-trifluoroethane, 2-Chloro-1,1,1,2-tetrafluoroethane, 1-Chloro-1,1,2,2-tetrafluoroethane, Pentafluoroethane, Pentafluorodimethyl ether, 1,1,2,2-Tetrachloroethane, 1,1,1,2-Tetrachloroethane, 1,1,2-Trichloro-2-fluoroethane, 1,1,2-Trichloro-1-fluoroethane, 1,1,1-Trichloro-2-fluoroethane, Dichlorodifluoroethane, 1,1-Dichloro-2,2-difluoroethane, 1,2-Dichloro-1,1-difluoroethane, 1,1-Dichloro-1,2-difluoroethane, 1,2-Dibromo-1,1-difluoroethane, 1-Chloro-1,2,2-Trifluoroethane, 1-Chloro-2,2,2-Trifluoroethane, 1-Chloro-1,1,2-Trifluoroethane, 1,1,2,2-Tetrafluoroethane, 1,1,1,2-Tetrafluoroethane, Bis(difluoromethyl)ether, 1,1,2-Trichloroethane, 1,1,1-Trichloroethane (Methyl chloroform), 1,2-Dichloro-1-fluoroethane, 1,2-Dibromo-1-fluoroethane, 1,1-Dichloro-2-fluoroethane, 1,1-Dichloro-1-fluoroethane, Chlorodifluoroethane, 1-Chloro-1,2-difluoroethane, 1-Chloro-1,1-difluoroethane, 1,1,2-Trifluoroethane, 1,1,1-Trifluoroethane, Methyl trifluoromethyl ether, 2,2,2-Trifluoroethyl methyl ether, 1,2-Dichloroethane 1,1-Dichloroethane Chlorofluoroethane, 1-Chloro-1-fluoroethane 1,2-Difluoroethane, 1,1-Difluoroethane Chloroethane (ethyl chloride) Fluoroethane Ethane Dimethyl ether, 1,1,1,2,2,3,3-Heptachloro-3-fluoropropane, Hexachlorodifluoropropane 1,1,1,3,3-Pentachloro-2,2,3-trifluoropropane, 1,2,2,3-Tetrachloro-1,1,3,3-tetrafluoropropane, 1,1,1-Trichloro-2,2,3,3,3-pentafluoropropane, 1,2-Dichloro-1,1,2,3,3,3-hexafluoropropane, 1,3-Dichloro-1,1,2,2,3,3-hexafluoropropane, 1-Chloro-1,1,2,2,3,3,3-heptafluoropropane, 2-Chloro-1,1,1,2,3,3,3-heptafluoropropane, Octafluoropropane , 1,1,1,2,2,3-Hexachloro-3-fluoropropane, Pentachlorodifluoropropane, 1,1,1,3,3-Pentachloro-2,2-difluoropropane, Tetrachlorotrifluoropropane, 1,1,3,3-Tetrachloro-1,2,2-trifluoropropane, 1,1,1,3-Tetrachloro-2,2,3-trifluoropropane, Trichlorotetrafluoropropane, 1,3,3-Trichloro-1,1,2,2-tetrafluoropropane, 1,1,3-Trichloro-1,2,2,3-tetrafluoropropane, 1,1,1-Trichloro-2,2,3,3-Dichloropentafluoropropane, 2,2-Dichloro-1,1,1,3,3-pentafluoropropane, 2,3-Dichloro-1,1,1,2,3-pentafluoropropane, 1,2-Dichloro-1,1,2,3,3-pentafluoropropane, 3,3-Dichloro-1,1,1,2,2-pentafluoropropane, 1,3-Dichloro-1,1,2,2,3-pentafluoropropane, 1,1-Dichloro-1,2,2,3,3-pentafluoropropane, 1,2-Dichloro-1,1,3,3,3-pentafluoropropane, 1,3-Dichloro-1,1,2,3,3-pentafluoropropane, 1,1-Dichloro-1,2,3,3,3-pentafluoropropane, Chlorohexafluoropropane, 2-Chloro-1,1,1,2,3,3-hexafluoropropane, 3-Chloro-1,1,1,2,2,3-hexafluoropropane, 1-Chloro-1,1,2,2,3,3-hexafluoropropane, 2-Chloro-1,1,1,3,3,3-hexafluoropropane, 1-Chloro-1,1,2,3,3,3-hexafluoropropane, 1,1,2,2,3,3,3-Heptafluoropropane, Trifluoromethyl 1,1,2,2-tetrafluoroethyl ether, 1,1,1,2,3,3,3-Heptafluoropropane, Trifluoromethyl 1,2,2,2-tetrafluoroethyl ether, Pentachlorofluoropropane, Tetrachlorodifluoropropane, 1,1,3,3-Tetrachloro-2,2-difluoropropane, 1,1,1,3-Tetrachloro-2,2-difluoropropane, Trichlorotrifluoropropane, 1,1,3-Trichloro-2,2,3-trifluoropropane, 1,1,3-Trichloro-1,2,2-trifluoropropane, 1,1,1-Trichloro-2,2,3-trifluoropropane, Dichlorotetrafluoropropane, 2,2-Dichloro-1,1,3,3-tetrafluoropropane-2-Dichloro-1,1,1,3-tetrafluoropropane, 1,2-Dichloro-1,2,3,3-tetrafluoropropane, 2,3-Dichloro-1,1,1,2-tetrafluoropropane, 1,2-Dichloro-1,1,2,3-tetrafluoropropane-3-Dichloro-1,2,2,3-tetrafluoropropane, 1,1-Dichloro-2,2,3,3-tetrafluoropropane, 1,3-Dichloro-1,1,2,2-tetrafluoropropane, 1,1-Dichloro-1,2,2,3-tetrafluoropropane, 2,3-Dichloro-1,1,1,3-tetrafluoropropane, 1,3-Dichloro-1,1,3,3-tetrafluoropropane-1-Dichloro-1,3,3,3-tetrafluoropropane, Chloropentafluoropropane, 1-Chloro-1,2,2,3,3-pentafluoropropane, 3-Chloro-1,1,1,2,3-pentafluoropropane, 1-Chloro-1,1,2,2,3-pentafluoropropane, 2-Chloro-1,1,1,3,3-pentafluoropropane, 1-Chloro-1,1,3,3,3-pentafluoropropane, 1,1,1,2,2,3-Hexafluoropropane, 1,1,1,2,3,3-Hexafluoropropane, 1,1,1,3,3,3-Hexafluoropropane, 1,2,2,2-Tetrafluoroethyl difluoromethyl ether, Hexafluoropropane, Tetrachlorofluoropropane, Trichlorodifluoropropane, ichlorotrifluoropropane, 1,3-Dichloro-1,2,2-trifluoropropane, 1,1-Dichloro-2,2,3-trifluoropropane, 1,1-Dichloro-1,2,2-trifluoropropane, 2,3-Dichloro-1,1,1-trifluoropropane, 1,3-Dichloro-1,2,3-trifluoropropane, 1,3-Dichloro-1,1,2-trifluoropropane, Chlorotetrafluoropropane, 2-Chloro-1,2,3,3-tetrafluoropropane, 2-Chloro-1,1,1,2-tetrafluoropropane, 3-Chloro-1,1,2,2-tetrafluoropropane, 1-Chloro-1,2,2,3-tetrafluoropropane, 1-Chloro-1,1,2,2-tetrafluoropropane, 2-Chloro-1,1,3,3-tetrafluoropropane, 2-Chloro-1,1,1,3-tetrafluoropropane, 3-Chloro-1,1,2,3-tetrafluoropropane, 3-Chloro-1,1,1,2-tetrafluoropropane, 1-Chloro-1,1,2,3-tetrafluoropropane, 3-Chloro-1,1,1,3-tetrafluoropropane, 1-Chloro-1,1,3,3-tetrafluoropropane, 1,1,2,2,3-Pentafluoropropane, Pentafluoropropane, 1,1,2,3,3-Pentafluoropropane, 1,1,1,2,3-Pentafluoropropane, 1,1,1,3,3-Pentafluoropropane, Methyl pentafluoroethyl ether, Difluoromethyl 2,2,2-trifluoroethyl ether, Difluoromethyl 1,1,2-trifluoroethyl ether, Trichlorofluoropropane, Dichlorodifluoropropane, 1,3-Dichloro-2,2-difluoropropane, 1,1-Dichloro-2,2-difluoropropane, 1,2-Dichloro-1,1-difluoropropane, 1,1-Dichloro-1,2-difluoropropane, Chlorotrifluoropropane, 2-Chloro-1,2,3-trifluoropropane, 2-Chloro-1,1,2-trifluoropropane, 1-Chloro-2,2,3-trifluoropropane, 1-Chloro-1,2,2-trifluoropropane, 3-Chloro-1,1,2-trifluoropropane, 1-Chloro-1,2,3-trifluoropropane, 1-Chloro-1,1,2-trifluoropropane, 3-Chloro-1,3,3-trifluoropropane, 3-Chloro-1,1,1-trifluoropropane, 1-Chloro-1,1,3-trifluoropropane, 1,1,2,2-Tetrafluoropropane, ethyl 1,1,2,2-tetrafluoroethyl ether, Dichlorofluoropropane, 1,2-Dichloro-2-fluoropropane, Chlorodifluoropropane, 1-Chloro-2,2-difluoropropane, 3-Chloro-1,1-difluoropropane, 1-Chloro-1,3-difluoropropane, Trifluoropropane, Chlorofluoropropane, 2-Chloro-2-fluoropropane, 2-Chloro-1-fluoropropane, 1-Chloro-1-fluoropropane, Difluoropropane, Fluoropropane, Propane, Dichlorohexafluorocyclobutane , Chloroheptafluorocyclobutane, Octafluorocyclobutane, (Perfluorocyclobutane), Decafluorobutane (Perfluorobutane), 1,1,1,2,2,3,3,4,4-Nonafluorobutane, 1,1,1,2,3,4,4,4-Octafluorobutane, 1,1,1,2,2,3,3-Heptafluorobutane, Perfluoropropyl methyl ether, Perfluoroisopropyl methyl ether, 1,1,1,3,3-Pentafluorobutane, Dodecafluoropentane (Perfluoropentane) , and Tetradecafluorohexane (Perfluorohexane).

The performance of latent heat exchange can be increased if (a) a liquid droplet or gas bubble or solid phase formed on the surface is highly mobile and can be removed quickly from the surface to minimize the thermal passivation of the active surface under consideration and (b) the thickness of these thermal barriers (droplets, bubbles, solid phase) can be kept small either by promoting quick removal or by promoting dropwise deposition instead of filmwise deposition on the active surface under consideration. When a droplet or a bubble or solid phase is nucleated and the size of it grows (e.g., by nucleation and growth), there is a minimum size (aka. critical droplet size, D_{crit}, or departure diameter) for the droplets or bubbles to spontaneously leave the surface owing to the gravitational force (for droplets and solid phase) or buoyancy force (for bubbles) overcoming the forces associated with the interfaces (i.e. surface tension, wettability, adhesion). Upon the departure of a droplet or bubble or solid phase from the surface, a fresh active surface is regenerated promoting new droplet/bubble/solid phase nucleation and growth, so that enhanced latent heat transfer can be achieved. For example, in the case of shedding droplets on an inclined surface, a shedding droplet can pick up other small droplets via coalescence along its pathway to further enhance the regeneration of fresh surfaces. Localized fast growth of droplets/bubbles/solid phase is preferred and often observed via coalescence, which is also known to occur preferentially along the topographically raised regions (e.g. convex regions) of a given surface due to focusing effect of diffusion flux of incoming phase before phase change. For simplicity, the behavior of phase-change materials is discussed referencing water condensation, but it is understood that the principles and components described herein apply to gas phase and solid phase condensation and heat exchange.

### Heat Exchanger/Condenser Design

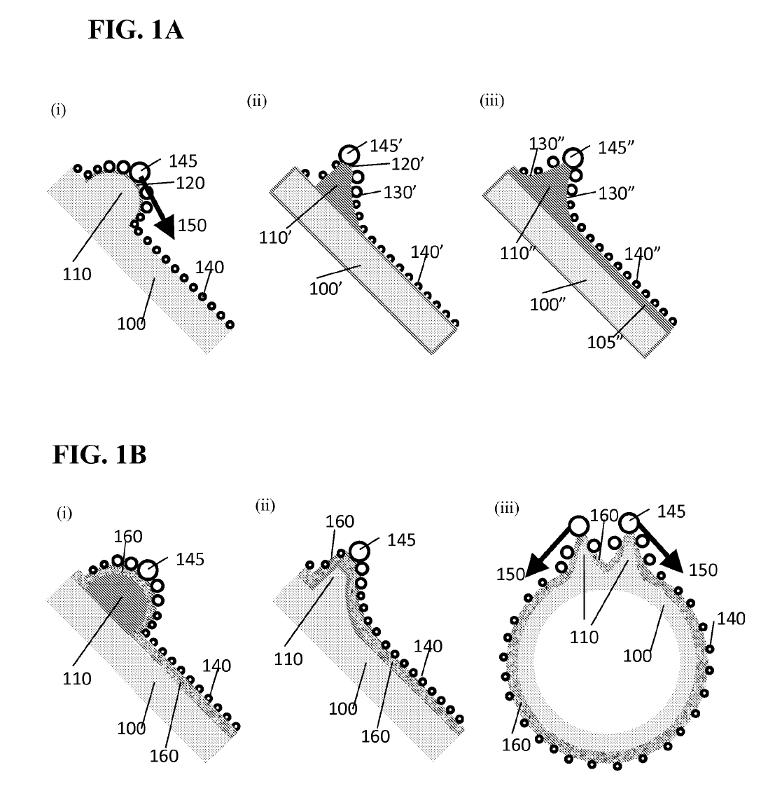

In one aspect of the invention, a SLIPS-based surface that is useful as a heat exchanger or condenser includes submicrometer to centimeter scale features on SLIPS-treated surfaces. Exemplary devices are illustrated in FIGS. 1A(i)-(iii), according to one or more embodiments. FIG. 1(i) shows a surface 100 having a raised convex feature 110 (shown here as a hemisphere). Droplet, bubble, or solid particle nucleation and growth starts at the features apex 120. Droplet growth is more efficient and droplets 145 formed the raised feature, and in particular near apex 120, are larger than droplets 140 formed on adjacent flat surface. When the particle reached a critical size, its sheds. In symmetrical systems such as in FIG. 1(i), shedding is non-directional. FIG. 1(ii) shows a surface 100′ having a raised convex feature 110′ and an asymmetric slope 130′ extending from apex 120′ and transitioning tangentially into the flat surface. Droplet bubble or solid particle nucleation and growth starts at the features apex 120′. Droplet growth is more efficient and droplets 145′ formed the raised feature, and in particular near apex 120′, are larger than droplets 140′ formed on adjacent flat surface. When the particle reached a critical size, its sheds directionally down slope 130′. FIG. 1(iii) shows a surface 100″ having a raised convex feature 110″ and an asymmetric slope 130″ extending from apex 120″ in more than one direction and transitioning tangentially into the flat surface. Droplet bubble or solid particle nucleation and growth starts at the features apex 120″. Droplet growth is more efficient and droplets 145″ formed the raised feature, and in particular near apex 120″ are larger than droplets 140″ formed on adjacent flat surface. When the particle reached a critical size, its sheds directionally down any one of slopes 130′. FIG. 1B(iii) shows a film 105 that is bearing the bumps and attached to the substrate.

In some embodiments, the device can be coated with a slippery surface that improves the shedding and droplet collection. Exemplary devices are illustrated in FIGS. 1B(i)-(iii), in which similar elements are similarly labeled, according to one or more embodiments. In each of these devices, a slippery layer 160 covers the device. The slippery layer can be a SLIPS coating. SLIPS coating provides functionalized nano/microasperities or nano/micropores that entrain a lubricating liquid, which fills void spaces, due to capillarity, and lock the lubricating liquid in and on the surfaces due to proper functionalization of the solid that provides chemical affinity to the lubricant, resulting in a molecularly smooth, slippery upper liquid surface. The underlying substrate need not be flat (e.g., an infinite radius of curvature), but can also be curved (but not that the radius of curvature for the raised features is smaller than that of the underlying substrate), as is shown in FIG. 1B(iii).

When used as a heat exchanger, the features and substrate can be made of thermally conductive components typically used in heat exchangers (e.g., aluminum, copper, stainless steel, etc.). In one or more embodiments, the latent heat exchanger includes a metal (or other thermally conductive polymeric or ceramic) substrate having a plurality of raised or recessed features. In one or more embodiments, the polymer or ceramic can include embedded metal to improve thermal conductivity. In one or more embodiments, embedded metal can be a metal mesh or metal particles.

When used as a condenser, the substrate and macro-scaled features are not required to be thermally conductive and can be made of polymers. The use of polymers, imparts the ability to create devices that are flexible and dynamically bendable or stretchable (deformable).

The raised or recessed features include a convex surface or an edge or rim feature on the surface that assists in nucleation and growth. The substrate containing the raised or recessed features contains a further hierarchy of surface treatment and includes a nano/micro-scale roughened surface and a lubricating liquid wetted and adhered to the roughened surface to provide a liquid overlayer within and over the roughened surface to form a repellant surface. The raised features can have a width and height in the range of 100 nm to 10 cm, or more specifically on the order of micrometers to centimeters, or more specifically on the order of hundreds of micrometers to tens of millimeters. In some embodiments, the features can have a height in the range of 100 μm to 5 cm, 0.5-2 mm or in the range of 1-10 mm. In one or more embodiments, the feature height is smaller than the depletion layer, H<δ (the dashed line denoted in FIG. 2C), so that diffusion is the dominant mechanism of mass transport. In some embodiments, the features can have a width on the dimension of the condensing droplets. In one or more embodiments, asymmetric structures have a scale that is similar to the shedding droplets (diameter ca. 0.6 mm). The features of the roughened surface are on a scale that are significantly smaller than the raised surface features used to condense and shed droplets.

The entire surface of SLIPS-based heat exchanger, including the raised or recessed features, is treated to form a SLIPS low friction surface (FIG. 1B(i)-(iii) and FIG. 17A-17E.). The SLIPS comprises functionalized nano/microasperities or nano/micropores (with the roughness factor from 1 representing a largely flat surface, to infinity representing a highly porous solid) that entrain a lubricating liquid, which fills void spaces, due to capillarity, and lock the lubricating liquid in and on the surfaces due to proper functionalization of the solid that provides chemical affinity to the lubricant, resulting in a molecularly smooth, slippery upper liquid surface (see FIG. 1B(i)-(iii)). FIGS. 17A-17E provide exemplary surfaces with immobilized lubricant overlayer: A) nano/microtextured surfaces with the roughness factor R>1; B) largely flat surfaces that utilize the natural roughness of the material (R=1); C) nano/microtextured surfaces with the roughness factor R>1 with the lubricant conformally coating the nano/micro-asperities.; D) nano/microtextured curved surfaces having a positive radius of curvature with the roughness factor R>1 with the lubricant conformally coating the nano/micro-asperities; and E) a nano/micro/milliscale flexible solid film layer 1770 (e.g., PDMS) that can hold and diffuse nano/microdroplets of lubricant. The features can be incorporated into a pipe structure and can be made on a region of the pipe (as shown) or over the entire surface. The elements are similarly labeled in each figure, indicating the substrate 1700; a nano/micro texture 1710; a functionalized interface 1720 that has high affinity to a lubricant (can be the natural chemistry of the substrate); a lubricant 1730; and a stabilized slippery liquid interface 1740 formed on the substrate.

The nano/micro-scale roughened surface with overlaying immobilized lubricating liquid is referred to as a SLIPS surface. See, International Appin. No. PCT/US12/021928 and International Appin. No. PCT/US13/50364, the contents of which are incorporated by reference, for additional information on preparation and function of SLIPS surfaces.

FIG. 4D is a profilometer image (oblique view) of a SLIPS-treated raised feature according to one or more embodiments. The raised feature (here an asymmetric bump having tangentially connected slope) on a scale of microns to millimeters (ca. 600 μm) is covered with a surface nanostructure containing nano-scale asperities (ca. 100-200 nm) that are infused with lubricant to create a molecularly smooth slippery coating.

Without being limited to any specific mode of operation, it is believed that droplets grow faster on the raised surfaces because the depletion layer or boundary layer or the distance from the solid surface to the liquid/vapor interface is thinner. Boundary layer thickness varies due to the raised features and is thinnest at the tips of the raised features. Vapor is transported to the solid surface by diffusion, but in the case of raised surface the boundary layer is thinned, so that droplet formation is sped up. Droplet shedding is enhanced due to the low friction of the SLIPS surface.

A heat exchanger or condenser according to one or more embodiments is illustrated in FIGS. 2A and 2C. FIG. 2A is an optical profilometer image of an exemplary convex raised structure or “bumps” that can be used for the SLIPS-based heat exchanger surfaces, according to one or more embodiments. In one or embodiments, the substrate has a plurality of raised features. In other embodiments, the substrate can have a plurality of recessed features. FIG. 2C shows a surface having an array of convex structures that can be used for the SLIPS-based heat exchanger, according to one or more embodiments. FIG. 2D shows time-lapsed images of droplets growing by condensation on the apex of the bumps (top row) compared to a flat region with the same height H (bottom row). The largest droplet in each series is denoted by dotted circles at each time point. Thus, raised features having convex surfaces demonstrate more rapid droplet condensation than flat surfaces.

In one or more embodiments, the structure can be based on a hemisphere as is schematically illustrated in FIG. 11. FIG. 11 is a cross-sectional schematic illustration of the convex structures showing the dimensions of height (H), radius (R_{bump}), radius of curvature (κ⁻¹) and periodicity (Pₚₐₜₜₑᵣₙ) of the features. The raised feature can be based on other curved surfaces, such as ellipsoids and cones. In one or more embodiments, the radius of curvature can be in the range of about 100 nm (˜nucleation size)to 10 cm (˜industrial pipe radii).

The convex macroscopic surface topography, e.g., having a positive radius of curvature κ_{—1}, such as the convex structures shown in FIGS. 2A, 2C and 11, is capable of controlling diffusion flux.

In one or more embodiments, the structure can be based on other geometries, such as cubes, rectangular prisms, cylinders, pyramids and the like. Exemplary structures are shown in FIG. 3C, and can include rectangular prisms, cubes, truncated cones, truncated pyramids, hemispheres, hemi-ellipsoids, hemicylinders and the like. In some embodiments, the raised features include edges 200 used for mass nucleation and growth, such as the rectangular geometry illustrated in FIG. 13. Edges, ridges or rims can be viewed as structures having a flat upper region 1300 bordered by rounded edges 1310 having a small radius of curvature around the perimeter. FIG. 13 is an infrared camera image (bottom) of the rectangular bump (top, optical camera image), which shows no distinct temperature difference between the top flat area of the rectangular bump and the surrounding basal region. The black or dark color in the infrared camera image along the edge of the rectangle is due to the scattering and different reflection of the curved region. FIG. 3D is an optical image of raised rectangular bumps with the same height, but having a range of different widths.

In one or more embodiments, the walls of the raised features can be sloped to create an inclined transition from the apex of the raised feature and continuing smoothly and tangentially to the substrate. By ‘tangential transition’ as used herein, it is meant that the slope curves gradually to a zero slope, without an abrupt change in angle (relative to the plane of the substrate) that would give rise to a negative radius of curvature or concavity. Surfaces with negative radius of curvature can pin droplets, such as is seen in FIG. 4F. The pinned droplets 700 are adjacent to the vertically inclined walls. Exemplary structures are shown in FIG. 4E (i)-(iv), and can include structures in which all or a portion of the side walls form inclined slopes or ramps 610 from the apex 620 to the substrate. The apex may include truncated upper surfaces to provide upper plateaus 630 and rounded edges 640 with high positive radius of curvature. The isotropic features like the two denoted by asterisks in FIG. 4E(i) can promote droplet removal in any direction, irrespective of the orientation in the gravity field and can therefore be placed randomly on any geometrically convoluted substrate. FIGS. 4E(ii)-(iv) show exemplary hemicylindrical convex structures with tangential connection to the surrounding substrate, which can be easily designed, manufactured at low cost, and significantly increase overall latent heat transfer efficiency, in consideration of downward gravitational force or air flow. FIG. 4E(ii) shows a hemicylindrical convex structure that is horizontally positioned on a vertically positioned flat surface. FIG. 4E(iii) shows a horizontally-positioned hemicylindrical convex structure along the side wall of a vertically positioned cylinder (or pipe or curved surface). FIG. 4E(iv) shows a hemicylindrical convex structure along the apex of a horizontally positioned cylinder. This horizontal hemicylindrical convex structure on a horizontally positioned pipe is designed in consideration of (1) fast growth on a smaller convex structure and (2) shedding droplets from the apex of horizontal pipes, shown in FIGS. 14A-G.

Both convex (e.g., spherical) and edged (e.g., rectangular) raised features that have functionalized nano/microasperities or nano/micropores with entrained and locked lubricating liquid in and on the surfaces (i.e., SLIPS) demonstrate improved droplet formation (faster nucleation and growth) over a traditional ‘flat’ SLIPS surface.

In one or embodiments, the latent heat exchanger or condenser includes a substrate having a plurality of raised or recessed features in combination with the slope that form an asymmetric feature to provide directional removal of droplets, bubbles or solids. The asymmetry can be achieved by a slope that extends outward from the feature apex in one direction. The asymmetry facilitates directed droplet movement, forced coalescence into larger droplets that can be easily removed in the gravity field, and rapid shedding. In one or more embodiments, the dimension of the asymmetric feature can be in the range of 100 nm to 10 cm, or more specifically on the order of micrometers to centimeters, or more specifically on the order of hundreds of micrometers to tens of millimeters. In some embodiments, the features can have a length or width in the range of 100 μm to 5 cm, 0.5-2 mm or in the range of 1-10 mm. In one or more embodiments, asymmetric structures have a scale that is similar to the shedding droplets (diameter ca. 0.6 mm).

As is discussed in greater detail herein below, an exemplary asymmetric feature can be a side wall having a width at the basal surface, which is greater than the width of the feature at its pinnacle, and the side wall increases in width or spans outward as it approaches the basal surface. The gradually increasing width of the side wall of the raised feature increases droplet growth and shedding because the asymmetric geometry induces directed motion of the droplets nucleated on the raised features, such that they are forced to coalesce and grow by absorbing other droplets along their moving path, resulting in the fast shedding of the heavy, large droplets in the gravity field. Such devices having asymmetric raised or recessed surface features can be alternatively referred to herein as “SLIPS-A”—slippery surfaces with asymmetric surface features.

In one or more embodiments, the asymmetric raised feature can include a gradually sloping or inclined ramp or wall that provides a transition from a high point of the raised features to the basal surface of the heat exchanger. The transitional feature or “ramp” is positioned to create an asymmetric structure; the ramp feature is preferably positioned on the surface, so as to direct the shedding droplet in a desired direction. FIG. 4A is a schematic illustration of an asymmetric feature illustrating the basis for directional shedding. The symmetric feature 300 (shown left) possesses four equivalent sides, or walls 310. Droplet 315 can shed in any of the directions noted by arrows 320. In contrast, asymmetric structure 330 shown in FIG. 4B includes a high point 340 and a ramp 350 that extends from the high point along one side to create an asymmetry in the profile. The ramp 350 is narrower where it meets the high point 340 than where it meets the basal surface 360. As the ramp approaches the basal surface 350, the width of the asymmetric feature increases, the height of the asymmetric feature decreases, and the asymmetric feature merges tangentially with the basal surface. Droplet 370 sheds preferentially in the direction noted by arrow.

FIG. 4H(i)-(iii) is a series of photographs of a SLIPS-A surface such as shown in FIG. 4B. The raised features have an upper surface 440 having a width of about 0.2 mm; a ramp 450 extends from the pinnacle towards the lower edge of the photographs, gradually increasing in width to about 1 mm. Droplets form at the upper surface of the asymmetric feature and move down along the ramp due to gravity and the ramp incline. The location of the ramp can serve to direct the droplets from the upper surface in a selected direction. The series of photos is a time-lapse series showing droplet condensation and shedding in a moist atmosphere. Faster time for shedding droplets is observed due to the topographically-preferential condensation, forced coalescence, and negligible friction of SLIPS-A. The arrow points at the shedding droplet on SLIPS-A, which grows and begins to move faster than the droplets on flat SLIPS (surrounding area). The state-of-the-art superhydrophobic surface (SHS) shown in FIG. 4G(iv) and a superhydrophobic surface that has a similar asymmetric feature (shown by dashed lines) (SHS-A) shown in FIG. 4G(v) do not show droplet shedding for >20× extended duration of experiment under identical conditions with droplets that reach at least 3 times larger radius before they begin to move, regardless of the presence of the asymmetric feature.

In one or more embodiments, the latent heat exchanger can include asymmetric features that rely on grooves or troughs recessed into a SLIPS treated surface to improve droplet formation and shedding. The groove or trough recess portions of the SLIPS-treated surface create edges so that the moist air perceives the planar features to be “raised” relative to the recessed grooves and condenses on the planar features faster.

FIGS. 3A-3B and 5 illustrate this principle of enhanced nucleation and growth due to raised features (edge elements) and asymmetric structure design.

FIG. 3A shows side and top views of raised features 520, illustrating flat upper surface 525, the edge features 530 and condensed droplets 540, 550. Droplets 540 growing along edges 530 are larger those growing on the flat surface 525 at the same time point. In one or more embodiments employing convexity effects that induce relatively higher growth rate of condensed droplets in terms of volume, all the dimensions—width (W), spacing (D), height (H), and length (L) can be greater than the critical shedding droplet diameter. In one or more embodiments, the width (W) or spacing (D) of grooves can be smaller than the critical shedding droplet size. In this embodiments, the time to reach the critical shedding droplet diameter can be reduced by forced coalescence that makes two adjacent droplets faster growing along the edges touch each other in a confined geometry defined by the distance between growing edges. FIG. 3B is a side view of two condensed droplets 540, 540′ growing on the edges 550. The growing droplets along two adjacent edges are about to touch, leading to a forced coalescence of the droplets.

FIG. 5 is an image of a SLIPS-treated surface having a number of recessed features, e.g., grooves 510. While making reference to a recessed groove, the principle applies equally to raised asymmetric features. At its widest and deepest point, groove 510 has a width (W) and a depth (−z) and adjacent grooves form a ridge 515 having a dimension defined by the spacing D(z). The groove width tapers to a narrow gap 520 along its length (L) as the groove becomes shallower, eventually becoming level and merging with the SLIPS surface (z=0). In one or more embodiments, the angle of asymmetric grooves can be sharp enough to easily release the droplets condensed in the grooves and the length of the structure (a function of the angle of asymmetric grooves) can be sufficiently long so that the residual droplets near the tail of the grooves can also be removed by the shedding droplets. The width of the ridge 515 is at its narrowest at the mouth 560 of the groove and increases along length L. In operation, droplets 570 formed at the top of ridge 515 coalesce to form larger droplet 570, which continues to grow (by coalescence with smaller droplets) as it continues along the ridge. Gradually increasing width of ridge increases droplet growth and shedding because the shedding droplets grow by absorbing other droplets along their moving path. See, e.g., droplet 570.

FIG. 6 shows the formation and shedding of condensed water droplets on SLIPS (left) and SLIPS-A (right), in which regularly spaced grooves are located at the upper edge of the sample. Droplet shedding performances is summarized in Table 1, where frequency (mHz=10⁻³ Hz) refers to the number of droplets formed per second; diameter (mm) refers to droplet size upon shedding and volume flow rate (mL/hr/m²) refers to the volume of shedded droplets collected in an hour in a 1 m² area, passing the bottom horizontal dotted line 600 in FIG. 6.

TABLE 1
Results of the image analysis of shedding droplets on SLIPS and SLIPS-A.

| | SLIPS | SLIPS-A |
|---|---|---|
| Avg. Frequency (mHz) | 8.9 | 11.1 |
| Avg. Diameter (mm) | 1.92 | 2.27 |
| Avg. Volume Flow Rate | 43.2 | 77.4 |
| (mL/hr/m²) | | |

The values are averaged for the 180 images recorded for the last 30 minutes of 2 hour long experiments. The averaged volume flow rate is calculated with the measured water contact angle of 105° on the Carnation mineral oil (Sonneborne) used as a lubricant. As shown in Table 1, the SLIPS-A sample exhibits higher average frequency and diameter of shedding droplets compared to ‘flat’ SLIPS. This results in nearly 80% increase in average volume flow rate of condensed water droplets passing the horizontal line 600 shown in FIG. 6.

To further examine the effect of increased amount of condensed water (SLIPS-A) on latent heat transfer, overall heat transfer coefficient (OHTC) was measured using a serpentine-shaped setup and conditions shown in FIG. 7. These measurements show more than 100% enhancement in OHTC compared to typical superhydrophobic surfaces (expected to be a promising candidate for enhanced heat transfer), implying the extremely promising potential of SLIPS-A for a broad range of applications.

In one or more embodiments, a plurality of raised structures of the same geometry and feature sizes or of slightly different geometry and feature sizes on SLIPS-A can be arranged in a manner that the features form a row or an array where adjacent features in the same row or in the neighboring rows can be aligned (eclipsed) or staggered to cover the surface of heat exchangers. In one or more embodiments, a plurality of raised features on SLIPS-A can be placed on a tubular object, such as pipes or tubes, as well as on a fin-like object, such as cooling fins, in a similar arrangement described above to cover the surface of the tubular or fin-like object and induce accelerated directional shedding of the droplets. In one or more embodiments, the coverage of such a set of raised features can be uniform or randomly distributed over the heat exchanger surfaces. In one or more embodiments, the coverage of such a set of raised features can exist in a pattern on the heat exchanger surface to induce rapid condensation, forced coalescence, and shedding only on a given area. Any combination of the above arrangements is possible.

In one or more embodiments, a plurality of features is arranged on a surface to increase condensation efficiency across a preselected area. As shown FIGS. 5 and 6, the features can be arranged in a row. In other embodiments, a number of rows can be employed. The features can be aligned in rows and columns, as illustrated in FIG. 2C and 4K. The features can be located in row that are staggered or offset to increase coverage of condensation run-off. In one or more embodiments, the density of features is selected to optimize condensation efficiency. Referring to FIG. 11, in which the feature size is described in terms of radius of curvature of the feature R_{bump} and feature to feature spacing Pₚₐₜₜₑᵣₙ, it has been observed that at less than a critical value (e.g., Pₚₐₜₜₑᵣₙ/R_{bump}<2.5) the raised features compete for condensation and no additional enhancement in condensation is observed. Thus in determining the features density for a feature with a given R, improvements in condensation can be achieved by approaching a Pₚₐₜₜₑᵣₙ/R_{bump} of about 2.5 or greater than 2.5.

These SLIPS with convex asymmetric features (SLIPS-A) offer transformative technology that allows for significant reduction of energy consumption and for increased functional efficiency of various phase change handling applications, e.g., condensation systems.

The heat exchanger surface according to one or more embodiments possesses a variety of features to achieve fast growth and shedding of droplets. First, higher growth rate in terms of droplet volume is observed on the convex regions of surface features, frequently observed in nature such as on insect wings and referred to as a ‘convexity effect.’ Second, forced coalescence can be achieved by making two adjacent droplets to touch each other. The convexity effect and forced coalescence can make droplets to grow faster to reach the critical shedding droplet size. Lastly, shedding droplets can be guided using gradually increasing width of the raised feature because directional movement of the shedding droplets induces fast droplet growth due to the coalescence with other droplets along their moving path and resulting in increased velocity of the shedded condensate. While each of the surface features of the heat exchange surface described herein contribute to rapid growth and shedding of droplets, combining the described features of the multi-scale surface structures provided greater enhance and improved droplet growth and transport. Therefore, combinations of the features described individually are contemplated with the scope of the invention.

The aforementioned principles can also be applied for the bubble formation in the process of boiling liquids. In this case, the buoyancy force is “upward”; therefore we can consider the direction opposite to the direction of gravitational force for the bubble motion, once the bubbles reach the critical size that balances the buoyancy force and pinning force. On top of the frictionless mobility, yet another essential benefit of SLIPS and SLIPS-A compared to typical surfaces with extreme wettabilities (e.g., superhydrophobic, superoleophobic, superhydrophilic or superoleophilic surfaces) is that the contact angle of liquid is more or less 90°, which shows extremely low pinning behavior for both droplets and bubbles at the same time. On the other hand, superhydrophobic or superoleophobic surfaces show extremely high pinning force for vapor bubbles because the “contact angle” of bubbles is nearly 0°, forming “film” of vapor that significantly lowers heat transfer performance in boiling.

### Device Fabrication

The substrate can made of a variety of materials capable of introducing the raised or recessed features disclosed herein.

In some embodiments, the underlying substrate is a thermally conductive base, and can be made, for example, of metal (copper, aluminum, stainless steel, titanium alloy, etc.), conductive polymer, conductive ceramic or other thermally conductive surfaces for heat exchangers. The base is molded into various shapes including tubes, fins, shells, etc. In other embodiments, the substrate is not required to be thermally conductive. For example, condensation can be achieved by condensation without thermal conduction as a primary mechanism. For example, the heat may be transferred by radiation. The underlying case can be a non-conductive polymer or cement.

A raised or recessed feature can be formed by conventional mechanical/material manufacturing process ranging from pressing, to casting, imprinting, molding, hydroforming, rolling, extrusion, expansion, notching, stamping, embossing, welding, bonding, engraving, machining (e.g. cutting, laser cutting, water jet cutting), etching, 3D printing, and so forth.

Once a metal-containing surface is formed, the metal-containing surface can be chemically modified to form a surface structure with proper feature sizes, volume, density, and morphology, suitable as a porous surface for SLIPS. The nano/micro-structured surface used to create SLIPS can be made by any method. Suitable methods have been previously described and include optional chemical functionalization to render the roughened surface compatible with the lubricating liquid. See, e.g., International Appin. No. PCT/US12/021928 and International Appin. No.PCT/US13/50402, contents of which are incorporated herein in their entirety.

Any roughening processes known in the art may be used. Exemplary processes for roughening include application of liquid phase material (paint or ink, spray, spin, dip, air brush, screen printing, inkjet printing), deposition or reaction of gas phase material (CVD, plasma, corona. ALD, PVD), etching, spraying, sputtering or evaporation of metal or metal oxide, composite phase material deposition (particle+binder), electrodeposition or other solution phase growth of material (conducting polymer, electroplated metal, electrophoretic deposition of particles, surface-initiated polymerization, mineralization), gas phase growth of material (nanofibers), multiple layer deposition (repeated coating by layer-by-layer deposition), self-assembly of precursor material (minerals, small molecules, biomolecules, polymers, nanoparticles, colloids), or growth of layers by oxidation-transfer coating and printing (contact printing, pattern transfer). Base materials without additional roughening that exploit the natural roughness of the material (R=1) followed by chemical functionalization and application of the lubricant layer can be used as well (see FIG. 1B)

In one or more embodiments, metal surfaces can be transformed into porous surfaces having a high roughness factor by chemical treatment such as wet chemical reaction, hydrolysis, alcoholysis, solvolysis, acid-base reactions, hydrothermal or solvothermal reactions, electrochemical deposition or etching, oxidation, plasma etching, chemical vapor deposition or atomic layer deposition, sol-gel reaction, and the like. An exemplary surface roughening process includes the transformation of the surface layer of aluminum into a highly nanoporous boehmite. In some embodiments, a roughened surface based on a metal-containing compound can be fabricated directly on a pure metal substrate (e.g., a bare aluminum plate). In some embodiments, a roughened surface based on a metal-containing compound can be fabricated on a thin metal film created on a metal or nonmetal substrate. The thin metal film can be deposited on the substrate using conventional methods such as vapor deposition (chemical vapor deposition (CVD). atomic layer deposition (ALD), physical vapor deposition (PVD), etc.), sputter deposition, electron beam evaporation, electro or electroless plating, and the like. In some embodiments, a roughened surface based on a metal-containing compound can be fabricated on a metal containing solution-based mixture (e.g., sol-gel coating) deposited on a metal or nonmetal substrate. The solution-based mixture can be applied by various application methods including dipping, spraying, painting, etc. Once formed, the metal layer is reacted, e.g., with water, air, alcohol or acid, to form a nanostructured oxide or oxyhydroxide. Further details for the formation of a nanostructures metal-containing surface can be found in International Appin. No. PCT/US13/50364, contents of which are incorporated in their entirety by reference.

Once the desired surface micro- or nano-structure is formed, it can be further chemically functionalized to provide the desired chemical affinity for the lubricating liquid. For example, the resulting structured surfaces can be further functionalized for appropriate compatibility with the lubricating liquid (e.g., using silane, thiol, carboxylate, phosphonate, phosphate, etc. as a reactant).

The lubricant layer is desirably immiscible with the operating materials used for phase change. In one or more embodiments, the lubricating liquid can be a hydrophobic liquid, as mineral oil, silicone oil or hydrocarbon oils. In other embodiments, the lubricating liquid can be an omniphobic liquid, such a fluorinated and perfluorinated oils.

The lubricating layer may serve as an additional thermal resistance layer on the latent heat exchanger. The effect of an additional lubricant layer should be considered in terms of thermal conductivity, thickness, surface tension, viscosity, boiling/melting point, toxicity etc. In one or more embodiments, the lubricant is selected to have a surface tension ranging from 1 mN/m to 1 N/m. In one or more embodiments, the kind of lubricant is selected to have viscosity ranging from 10⁻⁶ Pa·s to 10⁶ Pa·s. In one or more embodiments, the lubricant is selected to have boiling point higher than the operation temperature of phase change-based devices. In one or more embodiments, the lubricant is selected to have melting point lower than the operation temperature of phase change-based devices. In one or more embodiments, the kind and thickness of a lubricant is selected to enhance or at least not impair thermal conductivity. In one or more embodiments, the lubricant is selected to have high thermal conductivity, so that the thermal conductivity of the underlying thermally conductive substrate remains accessible. In one or more embodiments, the lubricant can be chosen so that it does not form a wrapping layer on the condensed liquid droplets that remove the lubricant when shed from the surface. Examples include various white mineral oils, poly(alphaolefin), polyalkylene glycol, and modified silicone oil, whose surface tension ensures infusion into nano/microtextured surfaces and the formation of the surface overlayer, but prevents the formation of the complete wrapping layer of condensed droplets. These lubricants can have a range of suitable viscosities, immiscible with the phase-change fluid, high viscosity index, low volatility, low toxicity, low flammability, and low cost. White mineral oil (e.g. Carnation from Sonneborn), which has approximately two times greater thermal conductivity value than fluorinated Krytox lubricants, can be used. The lubricant layer can be spincoated (e.g. at the angular velocity of 2000 rpm), on the nano/micro-structured surface (e.g. on boehmitized, nanostructured aluminum surfaces) to minimize the overall thermal resistance. A rough calculation shows that its overall thermal resistance is equivalent to only 25% of the overall thermal resistance introduced by the water layer on SHS.

Together with the selection of lubricant and hierarchical structure design, one can facilitate the formation of droplets or bubbles or solid phase with nearly frictionless mobility.

### Applications

Energy-efficient heat transfer through phase change is critical in numerous applications involving thermal to thermal energy conversion such as thermal power plant condensers, harvesters, desalination plants, distillation towers (e.g., hydrocarbons, polyolefins, hydrofluorocarbons), and building thermal/humidity control systems, vapor deposition systems. The latent heat transfer properties of the current invention can provide energy-saving solutions for water harvesters for drinking and irrigational water, multi-stage flash (MSF) desalination plants, vehicle/building air conditioners (i.e., HVAC systems), dehumidifiers, and oil refinery plants (e.g., hydrocarbons, polyolefins, hydrofluorocarbons) that are currently associated with significant energy penalties.

Thermal power plants are among the most dominant electricity generation facilities in the US. Furthermore, building temperature/humidity control systems and distillation towers use ˜15% and 6% of total energy in the US, respectively. On current thermal power plant condenser surfaces, the growth of droplets to reach the diameter of spontaneous removal (D_{crit}) is rather slow due to low rate of vapor diffusion and subsequent coalescence, and the spontaneous shedding of strongly pinned condensates can only happen when the droplet size grows larger, e.g., larger than 5 mm. As a result, thick, continuous, and thermally insulating condensate films or large droplets persist on the low-temperature walls which makes the overall heat transfer inefficient and results in the increase of tremendous energy input, increased greenhouse gas emission and increased use of cooling water.

a. Increased Efficiency in Thermal Power Plant Condensers

For thermal power plant condensers, high condensation efficiency results in operating the condensers at lower pressures and hence increasing the “pressure drop” of the saturated steam across a turbine, which translates into a potential improvement in energy generation of 20 million MWh/year, equivalent to a net savings on the order of $2 billion yearly (assuming a price of $0.1/kWh).

In a thermal power plant, fossil fuel is converted to electricity by burning the fuel to boil water to generate steam that drives a turbine attached to a generator. The steam that has passed the turbine is cooled and condensed back to water in a condenser unit, which is then pumped back to a boiler where the fuel is burning. The entire process happens in a closed loop. By lowering the temperature of the steam that has passed a turbine, the latent heat exchanger of the current invention can increase the pressure difference to drive the turbine with higher efficiency. This can be achieved by cooling the steam into tiny water droplets on a surface that is continuously maintained at a cold temperature by pumping cold water inside (i.e. the condenser unit), then removing those tiny droplets as quickly as possible to expose fresh and cold surface for the incoming hot steam. SLIPS-A enables efficient phase transformation (i.e. condensation of water vapor to liquid) to cool down the steam temperature while keeping the condensed water warmer which must be heated again at the boiler by burning fossil fuel to generate high pressure steam.

To estimate the enhancement of thermal power plant efficiency, which is the source for calculating the potential money and energy saving, we should first understand Rankine cycle, the theoretical model for designing thermal power plants.

The steam cycle of thermal power plants is composed of the four parts. High temperature steam with high pressure generated from the boiler (Q_{H}, 2→3) rotates the turbine (W_{T}, 3→4). While rotating the turbine, the saturated steam expands, resulting in lower temperature and pressure. It undergoes a phase change from vapor to liquid in the condenser (Q_{L}, 4→1) by heat exchange with coolants. The condensed water is pumped (W_{P}, 1→2) to the boiler and then reused to make this thermodynamic cycle.

The theoretical efficiency of this thermodynamic cycle, called Rankine cycle, can be calculated by the ratio between the area hatched with solid line (W net=Q_{H}−Q_{L}) and the area hatched with dotted lines (QH), shown in FIG. 16A. Therefore, to increase this efficiency, we can lower the temperature (T₄→T₄′) of saturated steam transported from the turbine to the condenser based on the comparison of cycle 1234 with cycle 1′2′34′ in FIG. 16B. Physically, this lower temperature leads to lower pressure (P_{4′}<P₄) of the saturated steam and thus greater pressure difference across the turbine, resulting in more driving force to rotate the turbine blades and theoretical efficiency improvement. Currently, the performance of condensation is plagued by filmwise condensation, which makes the temperature (T₄) of saturated steam relatively high. By introducing high condensation efficiency material or coating (SLIPS-A), one can effectively lower this temperature further.

b. Correlation Between the Low Pressure of Saturated Steam Leaving the Turbine (or Entering the Condenser) and Condensation Performance of the Surface of Condenser

The very low pressure (P₄ and P₄′) of the saturated steam stems from the huge change in the volume of water after phase change from vapor (or steam) to liquid (or condensate) in the process 4→1 or 4′→1′. Therefore, the higher the condensation efficiency at a given temperature, the lower the pressure of the saturated steam.

This relation is further analyzed and applied after considering the reported values of overall heat transfer coefficient (OHTC) in the literature. Assuming all the heat transferred from the coolant to the surface where condensation occurs is utilized for phase change, overall heat transfer coefficient is proportional to the condensation efficiency. Even traditional ‘flat’ SLIPS shows improved performance compared to other state-of-the-art coatings. SLIPS also presents the potential to lower the vapor pressure, which is the pressure of the saturated steam, for the same temperature and volume flow rate of the coolant. In traditional untreated materials and SHS, OHTC generally decreases as vapor pressure decreases; however OHTC of SLIPS at ˜2.1 kPa is higher than those of other two coatings at ˜2.8 kPa, which means that SLIPS can condense similar amount of steam at lower pressures. See, Xiao et al., SCIENTIFIC REPORTS |3:1988 ↑DOI: 10.1038/srep01988.

Traditional ‘flat’ SLIPS already provide ˜100% enhancement in OHTC compared to superhydrophobic surfaces (SHS). The application of asymmetric hierarchical structures according to one or more embodiments disclosed herein provides further significant improvement in the OHTC.

c. Reduced Amount of Cooling Water for Condensation

The enhanced condensation performance can reduce the amount of cooling water used in thermal power plants, which is typically nearby sea or lake water. In addition to the cost for cleaning the cooling water, it can reduce the electricity used for operating the cooling water pump and give more options for selecting location of the power plants. [U.S. Energy Information Administration, Many newer power plants have cooling systems that reuse water, 2014, http://www.eia.gov/todayinenergy/detail.cfm?id=14971#]

d. Reduced Energy Usage to Boil the Returning Condensate

Alternatively, the enhanced condensation efficiency of SLIPS-A can be used to keep the temperature of condensate higher, regardless of the type of operating coolants (e.g., water for water cooled condensers (WCC), air for air cooled condensers (ACC), hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, bromochlorofluorocarbons, silicones, hydrofluorocarbons, hydrofluoroolefins, brines, eurammon, azeotropic compound, and compounds of aforementioned refrigerants). This enables reusing the remaining heat in the condensate for boiling and thus saving fuel costs and the use of coolants.

e. A Solution for Marine and Microbial Fouling Problem

Many thermal power plants use seawater as cooling water in the condenser and marine fouling causes significant problems ranging from loss in efficiency of cooling water pump and condensers, to mechanical damage, further to the safety of the nuclear power plants, let alone the high maintenance cost. The surfaces can also improve corrosion resistance.

It has been shown that SLIPS prevent the formation of bacteria, algae, calcareous fouling, and inorganic build-up on its surfaces. The above-mentioned properties of slippery surfaces can be used in various applications, including multi-stage flash (MSF) desalination plants, thermal and humidity management systems for buildings, etc., liquid harvesting by facilitating the condensation of vapor, effective prevention of mechanical failure of underwater ship parts (e.g., motor screws) by the relief of impact of bubbles generated from cavitation, release of bubbles that hinder the transport of liquid in the pipe and release of biofouling and microbial fouling in pipes and HVAC systems.

The invention is illustrated by reference to the following examples, which are presented for the purpose of illustration and are not intended to be limiting of the invention.

### EXAMPLE 1

Superhydrophobic surfaces and SLIPS with asymmetric structures on the top parts of the vertically positioned surfaces were prepared using the following method, as illustrated in FIG. 8. First, a thin aluminum piece (60 mm×55 mm×0.16 mm, aluminum alloy 1100, Heatcraft) was flattened and patterned using the two molds which were prepared by 3D printing technique, a high throughput manufacturing method. After asymmetric millimeter-size structures were patterned, the pieces were immersed in the aqueous Alcojet® (Alconox, Inc.) cleaning solution (0.5 w %) for 50 min with sonication. The surfaces were rinsed in deionized water, followed by acetone rinse. Then the cleaned surfaces were immersed in the boiling water for 10 min to boehmitize the surface to create nanostructures. To make the superhydrophobic surfaces (showing the contact angle, CA=155±5°), the samples were subsequently immersed in a solution of an appropriate surface modifier (a perfluoroalkylphosphate ester, FS-100 or a potassium salt of cetylphosphate ester, CPE-K). To form a SLIPS coating, the latter superhydrophobic surfaces were infused with a white mineral oil (Carnation, Sonneborn) using spincoating step (at 2000 rpm for 1 min, resulting in lubricant thickness of ˜5 μm).

All the condensation experiments were conducted in the transparent humidity control chamber to maintain the desired relative humidity. All condensation experiments were done in a custom humidity chamber composed of a metallic frame with acrylic viewing windows and a door that enabled regulation of relative humidity (RH=60±5%) by a microprocessor controller (Model 5100-240, Electro-tech Systems, Inc.) and ultrasonic humidifier (AOS 7146, Air-O-Swiss) and surrounding ambient temperature (T=23±2° C.). The aluminum samples were mounted by using thermally conductive double-sided tapes (Thorlabs, TCDT1) on the serpentine shaped copper tubes (OD=¼ in), that are flattened to have a flattened width of =8.5±0.5 mm. Vertically positioned flat and bumpy test surfaces (T_{surface}=7.3±0.6° C.) were chilled through the thermal contact (3M™ Scotch Double Sided Conductive Copper Tape, 12.7 mm wide and 0.04 mm thick) with U-shaped copper tube (T_{tube}=2.3±0.3° C.). The temperature of surfaces and copper tube were measured with a digital thermometer.

The temperature and flow rate of coolant were controlled by a chiller (VWR 1167P or PolyScience 9106). The schematic illustration of the serpentine shaped copper tube structure and experimental conditions are shown in FIG. 7. The temperatures at the inlet and outlet of the copper tubing system were measured to quantify the overall heat transfer coefficients on the SLIPS and SLIPS-A compared to the SHS. The improvement of >100% for the OHTCs of SLIPS and SLIPS-A was confirmed in the experimental setup that satisfies relevant environmental conditions (humidity>85%, temperature <8° C.). To further examine the effect of increased amount of condensed water achieved by SLIPS-A, overall heat transfer coefficient normalized by the corresponding value for the SHS (OHTC/OHTC_{sHs}) was calculated based on the temperature measurement at the inlet and outlet of the serpentine-shaped copper tube setup. OHTC/OHTC_{SHS} is defined as,

$$\frac{OHTC}{{OHTC}_{SHS}} \approx \frac{{T}_{out} - {T}_{in} - Δ {T}_{baseline}}{{( {T}_{out} - {T}_{in} )}_{SHS} - Δ {T}_{baseline}} \qquad ( eq . 1 )$$

where Tₒᵤₜ and Tᵢₙ are measured temperature at the outlet and inlet, respectively, and ΔT_{baseline} is the Tₒᵤₜ-Tᵢₙ of the copper tube system without any mounted samples. Because the fluid properties such as specific heat, viscosity and density as well as temperature and flow rate of the coolant were identical for all experiments, the normalized OHTC value can be calculated according to eq. 1. As shown in the OHTC values normalized by SHS case (FIG. 10), these preliminary measurements show that SLIPS and SLIPS-A have more than 100% enhancement in OHTC compared to the superhydrophobic control. Due to the “flooded condensation”, SHS shows the lowest value, even compared to bare aluminum samples. Traditional ‘flat’ SLIPS exhibits ˜100% improved value because of the enhanced shedding properties facilitated by the friction-free liquid interface. SLIPS-A recorded the highest value (˜150% improvement), which shows an additional significant effect of the asymmetric structures on the droplet growth and shedding frequency. The error bars are from the intrinsic measurement error of resistance temperature detectors (RTDs, Omega, RTD-NPT-72-E-1/4, Class A) used to measure the temperatures at the inlet and outlet of the copper tube system.

The aluminum samples were imaged using the time-elapsed function of Canon EOS Rebel T4i (period of imaging=5 or 10 s). From the beginning of the experiments, the images were recorded for more than 2 h. Due to the nearly no shedding motion of droplets on the SHS, only shedding behaviors of condensed droplets on SLIPS and SLIPS-A were analyzed by using MATLAB codes developed based on Hungarian algorithm. Because the condensed droplets on the SHS are rarely moved, the condensation behavior of the SHS with asymmetric structures (SHS-A) was similar to that of SHS.

FIG. 6 shows the formation and shedding of condensed water droplets on SLIPS and SLIPS-A. The measured diameter is converted to the volume of droplets based on the measured contact angle of water droplets (CA=105°) and is reported in FIG. 9. Each shedding droplet passing the horizontal line 600 in FIG. 6 is represented by a vertical line on the time axis, with the height indicating the volume of the droplet. The bar graph represents the accumulated volume of shedding droplets passing the horizontal line 600. As seen in FIG. 9, even a rough, proof-of-concept design of SLIPS-A based on the consideration of the dimension range of interest exhibits higher frequency and volume of shedding droplets compared to SLIPS. This results in nearly 80% increase in total volume of collected condensed water droplets for the same period of time.

### EXAMPLE 2

To access the effect of raised surface features on droplet growth dynamics, hemispherical bumps precisely designed with three important geometrical parameters; radius of curvature, spacing ration, and height were evaluated, as shown in FIG. 11. The parameters of the geometrical design included hemispherical radius of curvature R_{bump}, spacing between the hemispheres Pₚₐₜₜₑᵣₙ and hemispherical patterning that can be captured by two dimensionless parameters r_{drop}/R_{bump} and R_{bump} /Pₚₐₜₜₑᵣₙ, where the r_{drop} refers to the radius of curvature for the condensed droplet.

The hemispherical bumps were fabricated by pressing aluminum foil (thickness ˜0.15 mm) between three-dimensionally printed polymer molds. To make the surface hydrophobic, liquid-phase surface treatment for 1 hour at 70° C. was followed after 20 minutes of cleaning using Alcojet® and acetone rinse. Water condensation experiments were done in the humidity chamber that enables the regulation of relative humidity at 60% and surrounding air temperature at 22° C. The test surfaces were positioned vertically and artificial humid airflow is introduced onto the surface.

Vertically positioned flat and bumped superhydrophobic surfaces were chilled through the contact with U-shaped copper tube at the temperature of 4° C. and the resulting condensation of water was captured by the sequential images as shown in FIG. 12. The center parts of FIG. 12 display water condensation on bumped hydrophobic surfaces whereas outer surroundings exhibit conventional dropwise condensation on the flat hydrophobic surface. Because of no shedding droplets by gravitation forces were observed (the critical shedding diameter>maximum droplet size observed in the experiment), the droplets grow on both surfaces by direct vapor to liquid phase change and coalescence. Due to the nearly uniform surface textures on flat surfaces, droplets grow following a power law on the temporal evolution of the average droplet diameter. In contrast, droplets on bumps grow much faster in the early stage and finally reach the similar diameter to the droplets on the flat surface.

The quantitative analysis on both flat and representative bumped surfaces for the time range from 500 seconds to 5000 seconds is provided in FIG. 12. Droplets on bumps grow much faster in the early stage resulting in a higher value for abscissa intercept and finally reach the similar diameter to the droplets on the flat surface, leading to gradually decreasing slope in the log scaled graph, whilst drops on flat regions show lower intercept and constant slope value in the range of observation time. In order to understand this unique droplet growth behavior on flat surface, droplet growth dynamics follows the power law suggested by Beysens et al. (r˜αt^{β}). Beysens' several studies have also revealed the difference of the coefficient alpha on the perpendicular edge of rectangular structures. However, any systematic study on the condensation characteristics of various geometries (e.g., radius of curvature at the edge, height and width of a convex structure) ranging from simple hemispherical bump to asymmetric features at millimetric length scale has not been reported to the best knowledge of the inventors.

Qualitatively, droplets grew faster and larger on the peaks of the hemispheres. By measuring the largest droplet diameters at logarithmically spaced time points and fitting a linear regression to the log-log plot of diameter vs. time, the time constant for growth on the bumps was calculated to be 0.60±0.04 or higher depending on experimental conditions.

### EXAMPLE 3

To optimize the focused diffusion flux, predictive models were developed that quantify the magnitude and spatial profile of vapor flux as a function of the radius of curvature. Models for steady state transport of dilute species were used to simulate the magnitude of diffusion flux. The depletion layer thickness (δ˜10 mm>H˜1 mm) was used. Axisymmetric coordinates and two-dimensional coordinates were used for spherical-cap-shaped bumps and rectangular bumps, respectively. The magnitude of the maximum diffusion flux focused at the apex of bumps does not decrease more than 5% if Pₚₐₜₜₑᵣₙ/R_{Bump}>2.5. See, FIG. 11. A plot of the simple scaling of diffusion flux near the apex of a spherical cap shows that the smaller the radius of curvature, the greater the localized diffusion flux. See, FIG. 2G.

Within a narrow region represented by the rectangle in FIG. 2F(i), the concentration gradient near the apex of spherical-cap-shaped bumps can be approximately estimated by converting the bump topography to a sphere with the same radius of curvature, as shown in FIG. 2F(ii) and calculating the concentration distribution created by a mass sink and its mirror image with the same magnitude and the opposite sign (i.e., corresponding mass source that has the same distance from its center to the depletion layer) as shown in FIG. 2F(iii). The concentration distribution at an arbitrary point on the line between the centers of the mass source and sink can be obtained by the superposition of two independent concentration distributions created by the source and sink, respectively. The models indicated that the area where the diffusion flux is greater than on the flat region with the same height becomes increasingly concentrated at and around the apex or the dome, and its maximum intensity grows stronger, as the radius of curvature decreases.

Consistent with the analytical and numerical models, the largest droplet diameters are experimentally observed at the apex of spherical-cap-shaped surface features that have the smallest radii of curvature (FIG. 2H), The radii of curvature for this investigation are set out in Table 2.

Table 2. Radius of curvature of various bumps used in FIG. 2H from the profilometer images.

TABLE 2
Radius of curvature of various bumps used in FIG. 2H
from the profilometer images.

| | Radius of curvature (mm) |
|---|---|
| Spherical-cap-shaped bump 1 | 4.2 |
| Spherical-cap-shaped bump 2 | 1.5 |
| Spherical-cap-shaped bump 3 | 0.53 |
| Rectangular bump | 0.18 |

However, the rate of droplet growth on the bump with the smallest radius of curvature begins to slow down at later time points (see upper curve in FIG. 2H). This change of slope suggests that the effect of the focused diffusion flux at the apex diminishes when this region becomes covered by the growing droplet.

### EXAMPLE 4

To maintain the advantages of the small radius of curvature but avoid the decrease in growth rate, a raised feature having a rectangular geometry for the apex was investigated, with a flat region on top bordered by rounded edges as shown in FIG. 3E. This shape incorporates an even smaller radius of curvature around the perimeter, combined with an additional area of focused flux on the top flat area. For a smaller width, the superposition of diffusion flux focused on these features collectively results in a larger contiguous area of high diffusion flux. Droplets on the rectangular structure, therefore, continue growing for a longer time at a constant growth rate, as shown in FIG. 3E, because the coalescence and moving of the growing droplets to the flat top area of the bump continues to provide fresh sites for re-nucleation and growth. FIG. 3E shows time-dependent droplet growth on bumps with decreasing width.

As the growing droplet does begin to cover the curved edges, the shape of the rectangular structure—flat with curved borders—also lends itself to a mechanism to transport the droplet directionally, when topographical asymmetry is integrated into the design. As previously shown, a droplet growing on a rectangular column will eventually fall off in a random direction. Adding a gradually widening slope descending from one side promotes downward motion by enabling the drop to transition to a completely flat surface. See, FIG. 4B. The total free energy of the droplet-bump-vapor system is lowest when the droplet is on a completely flat region of such an asymmetric convex structure where d=W, as illustrated in FIG. 4C. The upper portion of FIG. 4C shows the same droplet 370 of constant diameter at different positions as it moves along the slope 350 of asymmetric feature 330, with the corresponding normalized energy in the plot immediately below. At the far left, the droplet is larger than the flat area and overlaps with the low radius of curvature (rounded) edges, providing the normalized energy level for subsequent comparison. The capillary force resulting from this energy profile on a surface with negligible friction would lead the drop to move down the slope toward the wider flat area such that it will no longer overlap with the curved regions (shown as a dark edge band in FIG. 4C). The droplet shows the minimum energy state when the width of the flat region equals the diameter of the droplet (d=W), and therefore moves down the slope once it grows to cover the curved perimeter.

Asymmetric bumps with a tangential connection between the descending slope and the surrounding flat regions and having a slippery lubricant-locked nanocoating were fabricated (SLIPS-A). On the fabricated slippery asymmetric structures, droplets move even against gravity, as shown in FIG. 4F, because in such a system the capillary effect is dominant compared to gravitational effect (as captured by the Bond number—the ratio between gravitational force and capillary force−Bo=(ρ_{water}−μₐᵢᵣ)gr²/γ_{LV}˜1/7 at the length scale of the droplet, where ρ_{water} and ρₐᵢᵣ are the density of water and air, g is the gravitational constant, r is the radius of the droplet, and γ_{LV} is the surface tension of the water-vapor interface). The droplet shown by the arrow in the optical images partially covers the curved border (indicated by a higher reflection that can be seen as a thin bright region between the black sides and the grey flat top) of the asymmetric bump (t−t_{c}=−10 sec, where t_{c} is the time of completed coalescence). This drives it to move up to a wider region (t−t_{c}=−5 sec), where it then coalesces with another drop and moves further up along the bump (t−t_{c}=0 sec). The dotted line tracks the vertical progress of the droplet. Even though the modeling shown in FIG. 4C suggests that the lowest energy point for the moving droplet with a constant diameter is not the bottom of the bump (i.e., its widest region) and the droplet might be expected to be pinned upon reaching the point where d=W, the droplet can keep moving and accelerating as its size grows by coalescing with other small droplets on its path. This is observed by the droplet in FIG. 4F (indicated by an arrow) that merges with a second droplet (for example, as indicated by an asterisk), to form a larger droplet, which results in the condition d>W and drives the coalesced droplet to a location further down the slope, where the condition d=W is again satisfied. This mechanism guides droplet motion solely along the direction determined by the widening slope, regardless of the orientation of the bump relative to gravity. As a further example, FIG. 4G shows time-lapsed optical images of condensed water droplets on an exemplary asymmetric structure in combination with the SLIPS coating rotated 90° relative to gravity showing that the droplet travels solely in the direction determined by the bump geometry until it reaches the end of the bump and makes an immediate 90° turn to align with gravity. The dotted line tracks the horizontal motion of the droplet on the bump.

### EXAMPLE 5

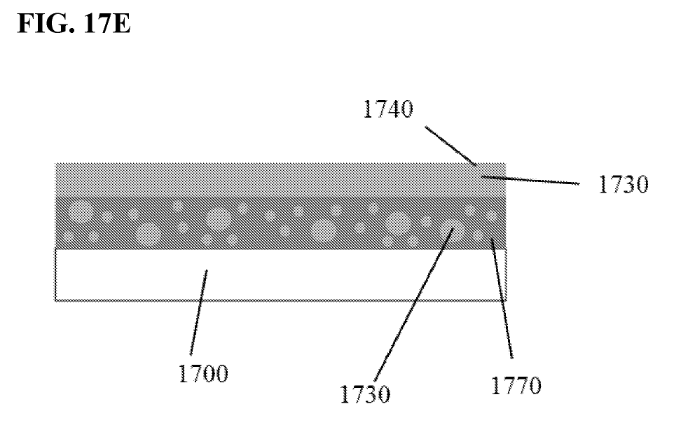

The surface structures according to one or more embodiments that include asymmetric bumps and SLIPS surfaces exhibit droplets that rapidly grow and are shed much earlier as compared to other state-of-the-art surfaces. FIG. 41 provides quantitative analysis of the droplet growth as a function of time, comparing the performance of slippery and superhydrophobic surfaces with and without asymmetric bumps. Both slippery asymmetric bumps (▴) and superhydrophobic asymmetric bumps (Δ) show faster localized droplet growth in the early stage (t<10³ sec) compared to flat slippery (▪) and superhydrophobic (□) controls. Drops on slippery asymmetric bumps exhibit a further enhanced growth rate (rectangular box in the middle of the plot) as they move down the bump slope, approximately six-fold greater than typical droplet growth dynamics, and the shortest t_{first} (i.e., the average time at which the first three drops are transported solely by gravity) of <˜20 min. Inset shows a magnified view of the fast growth, attributed to positive feedback between coalescence-driven growth and capillary transport. Neither superhydrophobic asymmetric bumps nor flat superhydrophobic surfaces produce shedding droplets within t˜200 min.

Flat-slippery surfaces outperform superhydrophobic surfaces in droplet size measured at a given time. Moreover, due to the aforementioned diffusion flux at the apex of the convex features, both slippery asymmetric bumps (solid black line) and superhydrophobic asymmetric bumps (dotted black line) show faster localized droplet growth in the early stage (t<10³ sec) compared to their flat controls. Surfaces with slippery asymmetric bumps show a unique discontinuous behavior, with a slope of ˜0.82 at the early stage and ˜6.4 at the later stage of droplet growth, which is more than six-fold higher than the maximum slope (˜1) observed in typical droplet growth dynamics

While each of the surface features of the heat exchange surface described herein contribute to rapid growth and shedding of droplets, combining the described features of the multi-scale surface structures provided greater enhance and improved droplet growth and transport. The accelerated growth (slope of 6.4) captured by the magnified view in the inset of FIG. 41 illustrates the feedback between coalescence-driven growth and capillary-driven transport discussed above. As a result, the fast growing droplets on the slippery asymmetric bumps, which are aligned with gravitational force, are delivered to the bottom of the slope at a size where they can then be transported by gravity, while droplets on the adjacent flat slippery surfaces are still far below the critical shedding diameter.

Droplets shed from the slippery bumps within t_{first}˜10³ sec, where t_{first} is the average time at which the first three drops are transported solely by gravity, whereas droplets on other state-of-the-art surfaces grow slowly, and shed much later (e.g. t_{first}˜4×10³ sec on flat slippery surfaces) or do not shed for more than t˜10⁴ sec (e.g. on superhydrophobic surfaces).

### EXAMPLE 6

An array of asymmetric features in an offset pattern was fabricated (see, FIG. 4K (left)) used the methods described above. The water collection capability was compared to a flat SLIPS surface (se FIG. 4K (right)). The offset placement of asymmetric raised features allowed for the shedding of one set of droplets without interference from nearby raised elements. The volume of collected water for these two surfaces over time is shown in FIG. 4L. Due to the faster droplet growth and transport performance of the SLIPS-A, which yields a continuous, rapid steady state turnover, a slippery surface with an exemplary array of the asymmetric structures showed an order of magnitude greater volume of water turnover compared to the flat slippery surfaces. FIG. 4M shows steady state water collection performance on bumpy (▴) and flat (▪) slippery surfaces. These extended data demonstrate that even with catastrophic shedding (which occurred at t=2 hrs on flat slippery surfaces) taken into account, the continuous turnover rate on the bumpy surface yields substantially more water over time. Shedding on the flat slippery surface does not show multiple shedding cycles because, after the initial catastrophic shedding, drops grow and shed randomly, rather than in periodic or predictable increments whereas the shedding pattern on bumpy surfaces is stable and uniform in time. In our experiments, the time-dependent water collection is intended not only as a measure of water production per se but also as a more general readout of the steady state turnover kinetics. This behavior is the reason synergy between growth and transport is so important. The fast turnover kinetics are established after just a few tens of minutes and then sustained continuously and indefinitely, yielding both a faster response time (time required to collect the first droplet) and an uninterrupted, reliable, and predictable performance over time. The constant faster shedding rate on the slippery surface with asymmetric bumps not only produces more total water but is crucial for many applications beyond simple water collection. This steady-state and predictable behavior is essential for applications such as phase change heat transfer, where a delayed or irregular performance could allow overheating or overcooling. Further, for water harvesting in arid regions, the faster response time is crucial because condensed water droplets will evaporate if they do not shed after a limited time.

### EXAMPLE 7

As a comparison, droplet growth curves were created for four surfaces previously developed for dropwise condensation, such as in heat exchange, dehumidification, and desalination systems. FIG. 4N shows droplet growth curve on four surfaces—macroscopic asymmetric bumps with nanostructure (without lubricant, A, slope=0.62), macroscopic asymmetric bumps with molecularly smooth lubricant (without nanostructure, ▴, slope=0.78), flat slippery coatings (without macroscopic bump, ▪, slope=0.79), and flat hydrophobic surfaces (as a control, ◯, slope=0.86). Whereas slippery flat surfaces (illustrated by curve ▪) showed shedding at t˜4×10³ sec, the other three surfaces (illustrated by curves Δ, ▴ and ▪) did not display shedding for more than t˜1.2×10⁴ sec. The slopes of all the lines are similar to each other because when condensed droplet size is smaller than the radius of curvature of the underlying convex structure, the effect of curvature does not significantly affect the droplet growth exponent.

### EXAMPLE 8

Water vapor condensation on slippery surfaces having raised convex structures without an asymmetric topography were evaluated for droplet growth and shedding. In one example, the raised features were hemispherical and had a radius of curvature in the range of 0.53-4.2 mm. FIGS. 40 and 4P (top) show images of droplets condensed on slippery spherical-cap-shaped bumps and rectangular bumps. FIGS. 4O and 4P (bottom) plot droplet diameter as a function of time for the hemispherical and rectangular prismatic features. The raised features generated condensed droplets of greater size than on the surrounding flat slippery surface. Even with a slippery coating, however, the bumps did not result in droplet shedding within the time studied, although the droplets were greater than the shedding diameter (denoted by the dotted horizontal line) measured on corresponding flat surfaces.

### EXAMPLE 9

The droplet growth rate near the apex and the base of the asymmetric raised features was evaluated. In this example, the fast growth and transport of droplets on slippery asymmetric bump apices were compared to the bottom edge regions. A plot of droplet size over time for slippery asymmetric bumps (▴), edge regions (▪) and flat slippery surfaces (▪) is shown in FIG. 4J. Each data point outside the three circles represents the average size of at least the three largest droplets on each surface. The three circles show the enhanced growth rate for the first three droplets that begin to move down by coalescence-driven growth and capillary-driven motion before leaving the bump and shedding solely by gravity. Each data point inside the three circles represents the diameter of the largest droplet on each of the bumps, obtained by tracking the same droplet on each of three different bumps. This plot demonstrates the superior drop growth behavior on the apex of bumps, compared to the bottom “edge” region.

### EXAMPLE 10

To apply the fast drop growth by the focused diffusion flux on convex surface curvature to another geometry that is widely used in industry, horizontally-positioned pipes with different diameters and the same aluminum thickness, hydrophobic coating, and surface temperature were tested. The quantitative results on the drop growth are shown in FIG. 14C. The pipes evaluated here can be viewed as the macro-scaled features with a positive radius curvature and, although they do not have slippery coatings, the difference in droplet size with pipe diameter (and thus radii of curvature) is an indication of the role surface curvature plays in droplet nucleation and growth. If the pipe surfaces are modified to become SLIPS, most of shedding droplets are observed to start from the top region of horizontally-positioned pipes regardless the diameter of pipes as droplet growth and transport are in steady-state. The shedding droplet volume on the smallest diameter pipe is the smallest because the center of mass of droplets growing on the smallest diameter pipe is more likely to be a position with a smaller elevation or altitude angle where a greater portion of gravitational force acts in the direction tangential to the underlying surface, compared to other greater diameter pipes. FIG. 14E is a plot of initial volume of shedding droplets (N=10), supporting the additional favorable effect of smaller pipe diameter on shedding droplet volume (or size), leading to earlier transport of condensed droplets.

FIG. 14D is a schematic illustration of a droplet on SLIPS pipe (cross sectional view) of different diameters, showing the effect of diameter of pipes on the magnitude of driving force (gravitational force's component that is tangential to the pipe surface, the length of darker arrows form the center of droplets) of droplet shedding. In the illustration, the size of droplet and the magnitude of gravitational force represented by the arrow pointing toward bottom are the same for the two different diameter pipes but the component of gravitational force tangential to the surface is greater on the smaller diameter pipe compared to the larger diameter pipe. This illustration explains why the size or volume of shedding droplets on a smaller diameter pipe is smaller compared to a larger diameter pipe.

As a result of enhanced drop growth and transport on a smaller diameter pipe, collected water in the container placed at the bottom of the pipes shows the greatest value in terms of the amount of water per unit area when the diameter of pipe is the smallest, as shown in FIG. 14F. A more collected water per unit area is a good indication of a higher heat transfer coefficient in phase change heat transfer because most of the heat transfer occurs in the process of condensation itself, compared to cooling down the temperature of the condensates.

Another example of a different radius of curvature geometry was evaluated, as shown in FIG. 14A at 3×10³ sec. In a cylindrical coordinate, the radius of curvature becomes smaller in the z-direction. FIG. 14B is a plot of droplet size as a function of z at t=1×10 ³ (), 2×10³ (♦), 3×10³ (▪), and 4×10³ (A) sec. Only the left end of the conical structure is thermally in contact with the low-temperature copper tube; therefore the temperature on the region with a smaller radius of curvature is higher than the region with a larger radius of curvature. Even against this unfavorable temperature gradient, the smaller radius of curvature regions showed larger droplets compared to the larger radius of curvature regions as shown in FIG. 14B, thus ruling out the role of temperature differences across the surface topography in directing the flux toward the apex of the bumps.

A further example is shown in FIG. 141, where a hierarchical structure is provided by forming raised bumps over a hemicylindrical (half-pipe) structure. (shown left). The size of the condensed droplets on the hierarchical structure is greater than that of the condensed droplets on a cylindrical geometry of similar radius of curvature (shown right) under identical condensation conditions.

### EXAMPLE 11

Longevity of SLIPS is essential for reliable enhanced performance of heat exchangers and other relevant applications. FIG. 14G shows an exemplary simple setup for significantly extending the lifetime of SLIPS on pipe geometry. The lubricant can be supplied to horizontal microchannels against gravity by capillary rise in oleophilic material. The horizontal microchannels are located on the top of the horizontal pipe to minimize undesired increase of critical shedding diameter by the microchannels perpendicular to the direction of drop shedding. The horizontal and vertical microchannels are designed based on the faster lubricant transport phenomena by microchannels confirmed in another experiment shown in FIG. 14H. The relatively bright regions along the grid of microchannels represent that the initially dry nanostructured regions are partially wetted by spreading of lubricant that moves along the microchannels faster than nanostructure only region. The cross sectional dimension of and distance between microchannels for replenishing lubricant can be chosen depending on the operating condition and rate of lubricant loss.

Microchannels that are aligned and perpendicular to the axis of the pipe and the oleophilic material facilitate transport of lubricant from reservoir by capillary effect. In particular, the width and depth of microchannel, as well as the spacing between microchannels can be tuned depending on the condensation condition (e.g., supersaturation, viscosity of lubricant at the pipe surface temperature). In multiple tests, at least an order of magnitude of longer lifetime has been confirmed and constant performance is anticipated as long as the reservoir is full of lubricant.

### EXAMPLE 12

Previous studies on condensation on topographically heterogeneous surfaces have found that the micro/nanoscale concave textures play a major role in preferential condensation if the textured surface is modified with a chemically homogeneous coating. To minimize the effect of the small length scale concave textures (e.g., less than the radius of curvature of the raised feature), the hemispheroidal surfaces were coated with PDMS. Polydimethylsiloxane (PDMS, 10:1 wt % of Sylgard 184 silicone elastomer base:Sylgard curing agent) was spun on the side of interest at 2,000 rpm for 2 min. The thickness of the deposited PDMS is 22.2±3.3 μm, calculated from the measurement of mass difference before and after the deposition. The PDMS-coated surfaces were examined using scanning electron microscopy (SEM) and profilometry. Whereas the uncoated surfaces exhibited microscale roughness, the PDMS-coated surfaces did not display the micro-roughness and were effectively smooth, as shown in FIG. 2B. The droplet growth on these surfaces is shown in FIGS. 2D and 2H.

To further compare the effect of the micro/nanoscale concave textures and that of millimetric convex topography on droplet growth in condensation, the same macroscopic geometry used in FIG. 2D was tested for droplet growth after roughening the flat surfaces with the 320 grit sandpaper before molding, cleaning and treating them with FS100 a perfluoroalkyl phosphate surface modifying agent. As shown in FIG. 2E, the bump without additional roughening by sandpaper shown still exhibited greater droplets on its apex compared to the roughened flat surfaces, thus ruling out the importance of the surface nano/micro roughness to the observed preferential droplet growth at the apex of the structures.

### EXAMPLE 13

The effect of an applied strain, termed ‘guided coalescence’ on a SLIPS-A surface was evaluated. In some embodiments, the substrate can be stretchable. For example, the substrate can be a stretchable polymer. The polymer can be poorly thermally conductive, thermally conductive or it can include embedded metal to improve thermal conductivity. For example, the metal can be metal particles or metal mesh. The ability to reversibly stretch the device allows for guided coalescence. Guided coalescence permits the controlled disconnection of the nucleation and growth process from the shedding process. By choosing to deform the substrate during a condensation process, it is possible to create asymmetry in the substrate that direct droplet (or bubble or sold) shedding at a time and in a direction that is guided by the deformation process.

FIG. 15A is a schematic illustration of drop movement and coalescence when underlying stretchable substrate is elongated in y-direction for a flat surface and a bumpy surface. Droplets fast grown on the apex of bumps by focused vapor diffusion flux are moved to concave regions between bumps, created by stretching a bumpy surface, and coalescence of these big droplets is also facilitated due to the suitable design of spacing between adjacent bumps. As a result, droplets not only grow faster on the apex of bumps by condensation but also grow even faster by guided coalescence. The cleaned apex of bumps can become fresh region for re-nucleation and re-growth of condensed droplets and growth/transport can be spatio-temporally controlled and enhanced by this “dynamic harvester” concept. FIG. 15B is a schematic illustration of the fabrication steps for creating a dynamic harvester. To enhance the thermal conductivity of the substrate, a copper mesh was used to create a composite structure material with soft polymers. Pre-stretching is an essential step to maximize the stretching capability of this composite material. FIG. 15C is an image of an exemplary dynamic harvester test setup according to one or more embodiments. FIG. 15D shows results of the condensation experiments using a dynamic harvester. Optical images show the predicted fast drop growth by (unstretched) bumps (a) and the effect of stretching the substrate on further growth by guided coalescence (b). The plot on the bottom right (d) shows gradual drop growth in the process of stretching and relaxing represented by strain-time plot (c).

Unless otherwise defined, used or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially, though not perfectly pure, as practical and imperfect realities may apply; e.g., the potential presence of at least trace impurities (e.g., at less than 1 or 2%) can be understood as being within the scope of the description; likewise, if a particular shape is referenced, the shape is intended to include imperfect variations from ideal shapes, e.g., due to manufacturing tolerances. Percentages or concentrations expressed herein can represent either weight or volume.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise.

It will be appreciated that while a particular sequence of steps has been shown and described for purposes of explanation, the sequence may be varied in certain respects, or the steps may be combined, while still obtaining the desired configuration. Additionally, modifications to the disclosed embodiment and the invention as claimed are possible and within the scope of this disclosed invention.

1. A phase change-based device comprising:
a substrate comprising a plurality of macro-scale raised or recessed features having a convex surface,
wherein the geometry of the feature promotes droplet, solid or bubble formation and accelerated growth on the apex of the raised feature, and removal of a phase of a phase-change material.

2. The device of claim 1, wherein the surface with a plurality of macro-scale features is coated with a slippery coating comprised of a lubricating liquid stably immobilized in and over the coating to promote accelerated removal of the phase change material.

3. The device of claim 1, further comprising a slope that transitions from an apex of the raised feature (or nadir of the recessed feature) tangentially to the substrate.

4. The device of claim 3, wherein the raised or recessed features in combination with the slope forms an asymmetric feature to provide directional removal.

5. The device of claim 3, wherein the raised or recessed features in combination with the slope forms a ramp around at least a portion of the raised feature to provide droplet, solid or bubble removal in more than one direction.

6. (canceled)

7. (canceled)

8. (canceled)

9. (canceled)

10. The device of claim 1, wherein the raised features comprise a cone, a hemisphere, a hemi-ellipse, a hemicylinder, pyramids, or bumps of irregular shape.

11. The device of claim 1, wherein the raised features comprise a flat upper surface and are selected from the group consisting of one or more of cubes, rectangular prisms, cylindrical columns, truncated cones, truncated pyramids and or truncated bumps of irregular shapes.

12. (canceled)

13. The device of claim 1, wherein the recessed features comprise a groove.

14. The device of claim 13, wherein the groove tapers from a first wide width to a second narrow width.

15. The device of claim 14, wherein the groove has an inclined floor that slopes upward tangentially to the substrate surface.

16. The device of claim 13, wherein the groove is in fluid contact with a reservoir holding lubricating liquid.

17. The device of claim 16, wherein the grooves are arranged to form a plurality of intersecting channels on the substrate.

18. The device of claim 1, wherein the features have a width in the range of 100 nm to 10 cm.

19. (canceled)

20. The device of claim 18, wherein the surfaces of the substrate and the macro-scale features comprise a nano-scale to micro-scale roughened surface.

21-44. (canceled)

45. The device of claim 1, wherein the device is in the shape of a pipe or coil.

46. The device of claim 1, further comprising a reservoir for supplying lubricating liquid to the device.

47. The device of claim 45, further comprising interconnected microchannel network to facilitate transport of lubricating liquid from a reservoir through the microchannels to the surface of the device.

48. The device of claim 1, wherein the features possess a radius of curvature R_{bump} of hemispherical features (or the width of asymmetric features W) and a feature to feature spacing Pₚₐₜₜₑᵣₙ and the features are positioned to provide a Pₚₐₜₜₑᵣₙ/R_{bump} (or Pₚₐₜₜₑᵣₙ/W) in the range of 1.1 to 100.

49. The device of claim 48, wherein P/R is in the range of 2.5-100.

50. The device of claim 1, further comprising a heat sink or coolant for removing adsorbed heat from the device.

51. The device of claim 1, wherein the device forms at least a portion of a phase change-based device in thermal power plant condensers, water harvesters, desalination plants, distillation towers (e.g., water, hydrocarbons, polyolefins, hydrofluorocarbons), building thermal/humidity, HVAC control systems, or vapor deposition systems.

52. A device for use in thermal power plant condensers, water harvesters, desalination plants, distillation towers (e.g., hydrocarbons, polyolefins, hydrofluorocarbons), building thermal/humidity control systems, or vapor deposition systems, wherein the device comprises the device of claim 1.

53. A method of condensing a phase change material on surface, comprising:
providing a device according to claim 1; and
exposing the heat exchanger to a form of a phase change material wherein the phase change material undergoes a phase change and heat is released or absorbed.

54-58. (canceled)

59. The method of claim 53, wherein the phase change material is water, polyolefins, hydrocarbons, propane, butane, ethylene, ethane, nitrous oxide, sulfur hexafluoride, dimethyl ether, halons, ammonia, sulfur dioxide, fluorocarbons, perfluorocarbons, chlorofluorocarbons, hydrochlorofluorocarbons, bromochlorofluorocarbons, perfluorocarbons, hydrofluorocarbons, hydrofluoroolefins, brines, eurammon, azeotropic compound, and refrigerants.

60. The method of claim 53, wherein the phase change material is a refrigerant and the material can be one or more of 1,1,1,2,2,3,3,4,4-Nonafluorobutane, Carbon tetrachloride (Tetrachloromethane), Trichlorofluoromethane, Dichlorodifluoromethane, Bromochlorodifluoromethane, Dibromodifluoromethane, Chlorotrifluoromethane, Bromotrifluoromethane, Tetrafluoromethane, Chloroform (Trichloromethane), Dichlorofluoromethane, Chlorodifluoromethane, Bromodifluoromethane, Trifluoromethane (Fluoroform), Dichloromethane (Methylene chloride), Chlorofluoromethane, Difluoromethane, Chloromethane, Fluoromethane, Methane, Hexachloroethane, Pentachlorofluoroethane, 1,1,2,2-Tetrachloro-1,2-difluoroethane, 1,1,1,2-Tetrachloro-2,2-difluoroethane, 1,1,2-Trichlorotrifluoroethane, 1,1,2-Trichlorotrifluoroethane, 1,2-Dichlorotetrafluoroethane, 1,1-Dichlorotetrafluoroethane, 1,2-Dibromotetrafluoroethane, Chloropentafluoroethane, Hexafluoroethane, Pentachloroethane, 1,1,2,2-Tetrachloro-1-fluoroethane, 1,1,1,2-Tetrachloro-2-fluoroethane, 1,1,2-Trichloro-2,2-difluoroethane, 1,1,2-Trichloro-1,2-difluoroethane, 1,1,1-Trichloro-2,2-difluoroethane, 2,2-Dichloro-1,1,1-trifluoroethane, 1,2-Dichloro-1,1,2-trifluoroethane, 1,1-Dichloro-1,2,2-trifluoroethane, 2-Chloro-1,1,1,2-tetrafluoroethane, 1-Chloro-1,1,2,2-tetrafluoroethane, Pentafluoroethane, Pentafluorodimethyl ether, 1,1,2,2-Tetrachloroethane, 1,1,1,2-Tetrachloroethane, 1,1,2-Trichloro-2-fluoroethane, 1,1,2-Trichloro-1-fluoroethane, 1,1,1-Trichloro-2-fluoroethane, Dichlorodifluoroethane, 1,1-Dichloro-2,2-difluoroethane, 1,2-Dichloro-1,1-difluoroethane, 1,1-Dichloro-1,2-difluoroethane, 1,2-Dibromo-1,1-difluoroethane, 1-Chloro-1,2,2-Trifluoroethane, 1-Chloro-2,2,2-Trifluoroethane, 1-Chloro-1,1,2-Trifluoroethane, 1,1,2,2-Tetrafluoroethane, 1,1,1,2-Tetrafluoroethane, Bis(difluoromethyl)ether, 1,1,2-Trichloroethane, 1,1,1-Trichloroethane (Methyl chloroform), 1,2-Dichloro-1-fluoroethane, 1,2-Dibromo-1-fluoroethane, 1,1-Dichloro-2-fluoroethane, 1,1-Dichloro-1-fluoroethane, Chlorodifluoroethane, 1-Chloro-1,2-difluoroethane, 1-Chloro-1,1-difluoroethane, 1,1,2-Trifluoroethane, 1,1,1-Trifluoroethane, Methyl trifluoromethyl ether, 2,2,2-Trifluoroethyl methyl ether, 1,2-Dichloroethane 1,1-Dichloroethane Chlorofluoroethane, 1-Chloro-1-fluoroethane 1,2-Difluoroethane, 1,1-Difluoroethane Chloroethane (ethyl chloride) Fluoroethane Ethane Dimethyl ether, 1,1,1,2,2,3,3-Heptachloro-3-fluoropropane, Hexachlorodifluoropropane 1,1,1,3,3-Pentachloro-2,2,3-trifluoropropane, 1,2,2,3-Tetrachloro-1,1,3,3-tetrafluoropropane, 1,1,1-Trichloro-2,2,3,3,3-pentafluoropropane, 1,2-Dichloro-1,1,2,3,3,3-hexafluoropropane, 1,3-Dichloro-1,1,2,2,3,3-hexafluoropropane, 1-Chloro-1,1,2,2,3,3,3-heptafluoropropane, 2-Chloro-1,1,1,2,3,3,3-heptafluoropropane , Octafluoropropane , 1,1,1,2,2,3-Hexachloro-3-fluoropropane, Pentachlorodifluoropropane, 1,1,1,3,3-Pentachloro-2,2-difluoropropane, Tetrachlorotrifluoropropane, 1,1,3,3-Tetrachloro-1,2,2-trifluoropropane, 1,1,1,3-Tetrachloro-2,2,3-trifluoropropane, Trichlorotetrafluoropropane, 1,3,3-Trichloro-1,1,2,2-tetrafluoropropane, 1,1,3-Trichloro-1,2,2,3-tetrafluoropropane, 1,1,1-Trichloro-2,2,3,3-Dichloropentafluoropropane, 2,2-Dichloro-1,1,1,3,3-pentafluoropropane, 2,3-Dichloro-1,1,1,2,3-pentafluoropropane, 1,2-Dichloro-1,1,2,3,3-pentafluoropropane, 3,3-Dichloro-1,1,1,2,2-pentafluoropropane, 1,3-Dichloro-1,1,2,2,3-pentafluoropropane, 1,1-Dichloro-1,2,2,3,3-pentafluoropropane, 1,2-Dichloro-1,1,3,3,3-pentafluoropropane, 1,3-Dichloro-1,1,2,3,3-pentafluoropropane, 1,1-Dichloro-1,2,3,3,3-pentafluoropropane, Chlorohexafluoropropane, 2-Chloro-1,1,1,2,3,3-hexafluoropropane, 3-Chloro-1,1,1,2,2,3-hexafluoropropane, 1-Chloro-1,1,2,2,3,3-hexafluoropropane, 2-Chloro-1,1,1,3,3,3-hexafluoropropane, 1-Chloro-1,1,2,3,3,3-hexafluoropropane, 1,1,2,2,3,3,3-Heptafluoropropane, Trifluoromethyl 1,1,2,2-tetrafluoroethyl ether, 1,1,1,2,3,3,3-Heptafluoropropane, Trifluoromethyl 1,2,2,2-tetrafluoroethyl ether, Pentachlorofluoropropane, Tetrachlorodifluoropropane, 1,1,3,3-Tetrachloro-2,2-difluoropropane, 1,1,1,3-Tetrachloro-2,2-difluoropropane, Trichlorotrifluoropropane, 1,1,3-Trichloro-2,2,3-trifluoropropane, 1,1,3-Trichloro-1,2,2-trifluoropropane, 1,1,1-Trichloro-2,2,3-trifluoropropane, Dichlorotetrafluoropropane, 2,2-Dichloro-1,1,3,3-tetrafluoropropane-2-Dichloro-1,1,1,3-tetrafluoropropane, 1,2-Dichloro-1,2,3,3-tetrafluoropropane, 2,3-Dichloro-1,1,1,2-tetrafluoropropane, 1,2-Dichloro-1,1,2,3-tetrafluoropropane 3-Dichloro-1,2,2,3-tetrafluoropropane, 1,1-Dichloro-2,2,3,3-tetrafluoropropane, 1,3-Dichloro-1,1,2,2-tetrafluoropropane, 1,1-Dichloro-1,2,2,3-tetrafluoropropane, 2,3-Dichloro-1,1,1,3-tetrafluoropropane, 1,3-Dichloro-1,1,3,3-tetrafluoropropane, 1-Dichloro-1,3,3,3-tetrafluoropropane, Chloropentafluoropropane, 1-Chloro-1,2,2,3,3-pentafluoropropane, 3-Chloro-1,1,1,2,3-pentafluoropropane, 1-Chloro-1,1,2,2,3-pentafluoropropane, 2-Chloro-1,1,1,3,3-pentafluoropropane, 1-Chloro-1,1,3,3,3-pentafluoropropane, 1,1,1,2,2,3-Hexafluoropropane, 1,1,1,2,3,3-Hexafluoropropane, 1,1,1,3,3,3-Hexafluoropropane, 1,2,2,2-Tetrafluoroethyl difluoromethyl ether, Hexafluoropropane, Tetrachlorofluoropropane, Trichlorodifluoropropane, ichlorotrifluoropropane, 1,3-Dichloro-1,2,2-trifluoropropane, 1,1-Dichloro-2,2,3-trifluoropropane, 1,1-Dichloro-1,2,2-trifluoropropane, 2,3-Dichloro-1,1,1-trifluoropropane, 1,3-Dichloro-1,2,3-trifluoropropane, 1,3-Dichloro-1,1,2-trifluoropropane, Chlorotetrafluoropropane, 2-Chloro-1,2,3,3-tetrafluoropropane, 2-Chloro-1,1,1,2-tetrafluoropropane, 3-Chloro-1,1,2,2-tetrafluoropropane, 1-Chloro-1,2,2,3-tetrafluoropropane, 1-Chloro-1,1,2,2-tetrafluoropropane, 2-Chloro-1,1,3,3-tetrafluoropropane, 2-Chloro-1,1,1,3-tetrafluoropropane, 3-Chloro-1,1,2,3-tetrafluoropropane, 3-Chloro-1,1,1,2-tetrafluoropropane, 1-Chloro-1,1,2,3-tetrafluoropropane, 3-Chloro-1,1,1,3-tetrafluoropropane, 1-Chloro-1,1,3,3-tetrafluoropropane, 1,1,2,2,3-Pentafluoropropane, Pentafluoropropane, 1,1,2,3,3-Pentafluoropropane, 1,1,1,2,3-Pentafluoropropane, 1,1,1,3,3-Pentafluoropropane, Methyl pentafluoroethyl ether, Difluoromethyl 2,2,2-trifluoroethyl ether, Difluoromethyl 1,1,2-trifluoroethyl ether, Trichlorofluoropropane, Dichlorodifluoropropane, 1,3-Dichloro-2,2-difluoropropane , 1,1-Dichloro-2,2-difluoropropane, 1,2-Dichloro-1,1-difluoropropane, 1,1-Dichloro-1,2-difluoropropane, Chlorotrifluoropropane, 2-Chloro-1,2,3-trifluoropropane, 2-Chloro-1,1,2-trifluoropropane, 1-Chloro-2,2,3-trifluoropropane, 1-Chloro-1,2,2-trifluoropropane, 3-Chloro-1,1,2-trifluoropropane, 1-Chloro-1,2,3-trifluoropropane, 1-Chloro-1,1,2-trifluoropropane, 3-Chloro-1,3,3-trifluoropropane, 3-Chloro-1,1,1-trifluoropropane, 1-Chloro-1,1,3-trifluoropropane, 1,1,2,2-Tetrafluoropropane, ethyl 1,1,2,2-tetrafluoroethyl ether, Dichlorofluoropropane, 1,2-Dichloro-2-fluoropropane, Chlorodifluoropropane, 1-Chloro-2,2-difluoropropane , 3-Chloro-1,1-difluoropropane , 1-Chloro-1,3-difluoropropane, Trifluoropropane , Chlorofluoropropane, 2-Chloro-2-fluoropropane, 2-Chloro-1-fluoropropane, 1-Chloro-1-fluoropropane, Difluoropropane, Fluoropropane, Propane, Dichlorohexafluorocyclobutane , Chloroheptafluorocyclobutane, Octafluorocyclobutane, (Perfluorocyclobutane), Decafluorobutane (Perfluorobutane), 1,1,1,2,2,3,3,4,4-Nonafluorobutane, 1,1,1,2,3,4,4,4-Octafluorobutane, 1,1,1,2,2,3,3-Heptafluorobutane, Perfluoropropyl methyl ether, Perfluoroisopropyl methyl ether, 1,1,1,3,3-Pentafluorobutane, Dodecafluoropentane (Perfluoropentane) , or Tetradecafluorohexane (Perfluorohexane).

61. The method of claim 53, wherein the phase change material forms as a liquid (droplet), gas (bubble) or solid.

62-67. (canceled)

68. The method of claim 61, wherein the heat exchanger is functional to effect condensation in multi-stage flash (MSF) desalination plants, thermal and humidity management systems for buildings, etc., liquid harvesting by facilitating the condensation of vapor, effective prevention of mechanical failure of underwater ship parts (e.g., motor screws) by the relief of impact of bubbles generated from cavitation, release of bubbles that hinder the transport of liquid in the pipe and release of inorganic and organic fouling.

69. A method of decoupling phase change material growth and transport;
comprising:
providing a phase change-based device a deformable substrate comprising a plurality of macro-scale raised features having a convex surface, wherein the geometry of the feature promotes droplet, solid or bubble formation and accelerated growth on the apex of the raised feature; and
condensing a phase change material on at last the apices of the macro-scale raised features or the device; and
deforming the substrate and the macro-scale raised features to remove a phase of a phase-change material from the apices.
